(19) United States
(12) Patent Application Publication
(10) Pub. No.: US 2024/0225682 A9
(43) Pub. Date: Jul. 11, 2024

(54) ULTRASOUND TISSUE TREATMENT APPARATUS

(71) Applicant: HEALIUM MEDICAL LTD., Yoneam (IL)

(72) Inventors: Ran Sela, Ramat Hasharon (IL); Yuri Megel, Nesher (IL); Dimitry Smakhtin, Kfar Yona (IL)

(21) Appl. No.: 18/547,220

(22) PCT Filed: Feb. 22, 2022
(86) PCT No.: PCT/IB2022/051547

§ 371 Date: Aug. 21, 2023

Related U.S. Application Data
(60) Provisional application No. 63/153,477, filed on Feb. 25, 2021.

(51) Int. Cl. A61B 17/32; A61B 8/00; A61B 8/08; A61B 8/12

(52) U.S. Cl. CPC A61B 17/320068; A61B 8/12; A61B 8/4466; A61B 8/483; A61B 2017/00743; A61B 2017/320069

(57) ABSTRACT

Apparatus (220/20) and methods are described including a transluminal ablation catheter (40) that includes at least one ultrasound transducer (50/52/150). The at least one ultrasound transducer is inserted into a chamber (190) of a subject's heart and is configured (a) to ablate tissue of the subject by applying ultrasound energy to the tissue, and (b) to image tissue of the subject by applying non-ablating ultrasound energy to the tissue. An expandable cage (30/301) is disposed around the at least one ultrasound transducer. The at least one ultrasound transducer is configured to rotate and axially translate back and forth within the expandable cage, such as to generate a three-dimensional image of the tissue. Other applications are also described.

### CROSS-REFERENCES TO RELATED APPLICATIONS

The present application claims priority from U.S. Provisional Patent Application No. 63/153,477 to Megel et al., filed Feb. 25, 2021, entitled “ULTRASOUND TISSUE TREATMENT APPARATUS AND METHOD”, which is incorporated herein by reference.

### FIELD OF EMBODIMENTS OF THE INVENTION

Some applications of the present invention generally relate to devices and methods for treatment of tissue by application of energy thereto, and more particularly to ablation of cardiac tissue by application of ultrasound energy for treating cardiac arrhythmias, such as atrial fibrillation.

### BACKGROUND

Atrial fibrillation is a common cardiac arrhythmia involving the atria of the heart. During atrial fibrillation, the atria beat irregularly and out of coordination with the ventricles of the heart, thereby disrupting efficient beating of the heart. Atrial fibrillation symptoms often include heart palpitations, shortness of breath and weakness. A major concern with atrial fibrillation is the potential to develop blood clots within the atria of the heart. These blood clots forming in the heart may circulate to other organs and lead to serious medical conditions such as strokes.

Atrial fibrillation is generally caused by abnormal electrical activity in the heart. During atrial fibrillation, electrical discharges may be generated by parts of the atria which do not normally generate electrical discharges, such as pulmonary vein ostia in the atrium.

Ablation procedures are generally used to terminate a faulty electrical pathway from sections of the heart, especially in those who are prone to developing cardiac arrhythmias and to restore the heart to its normal rhythm. For example, pulmonary vein isolation by ablation is a common medical procedure for treatment of atrial fibrillation.

### SUMMARY

There is provided in accordance with some applications of the present invention, apparatus for use with a lumen, e.g., a pulmonary vein, that extends from a heart chamber, e.g., the left atrium of the heart. Typically, the apparatus applies ultrasound energy to ablate tissue of the ostium of the pulmonary vein to electrically isolate the pulmonary vein to treat cardiac arrhythmia. For some applications, the apparatus comprises a transluminal ablation catheter comprising at least one ultrasound transducer coupled to a distal portion of the catheter and configured to be inserted into the chamber of the subject's heart, and to ablate the tissue of the ostium of the pulmonary vein by application of ultrasound energy. The transluminal ablation catheter additionally comprises a nipple-shaped expandable cage disposed around the ultrasound transducer. The nipple shape is typically formed by the expandable cage having a central portion having a diameter that is greater than a diameter of a distal portion of the expandable cage such that the distal portion of the expandable cage is shaped and sized to be inserted into the ostium of the pulmonary vein to temporarily anchor the distal portion of the catheter in the pulmonary vein by the contacting a wall of the vein.

For some applications, the expandable cage comprises a plurality of struts (e.g., flexible struts), which anchor the distal portion of the transluminal ablation catheter in the pulmonary vein by contacting the wall of the pulmonary vein. In some cases, the struts are referred to herein as “flexible wires”, with these two terms being used interchangeably throughout the specification and the claims. Typically, the struts are structured to form the expandable cage such that a plurality of relatively large gaps exist between the struts allowing blood flow and transmission of ultrasound energy through the expandable cage. Additionally, for some applications, at least a portion of the struts of the expandable cage are shaped to define one or more apertures formed in the struts, further facilitating transmission of the ultrasound energy to the tissue. For some applications, the apertures are formed along a length of the struts. The apertures formed in struts typically increase the area through which the ultrasound energy is transmitted through the expandable cage, thereby allowing for more ultrasound energy to reach the target tissue, resulting in increased heating of the tissue and more effective ablation of the tissue.

For some applications, in addition to the expandable cage, the apparatus comprises a fluid-filled inflatable element e.g., a balloon, disposed around the ultrasound transducer. Typically, the inflatable element is inflated with a fluid, such as water and/or saline, through which ultrasound energy is transmitted, but in which absorption of ultrasound energy is negligible. For such applications, the expandable cage is disposed around the inflatable element to position the distal portion of the transluminal ablation catheter in the lumen, as described hereinabove.

Typically, disposing the fluid-filled (e.g., water-filled) inflatable element around the ultrasound transducer, partially replaces the blood medium between the ultrasound transducer the and the tissue designated for ablation. Since ultrasound energy is transmitted through water but generally not absorbed by it, a greater amount of the transmitted ultrasound energy reaches the tissue in comparison to if the transmitted ultrasound energy were transmitted through only blood.

Additionally, or alternatively to applying ablative ultrasound energy, the ultrasound transducer is configured to image tissue of the subject by applying non-ablative ultrasound energy. Typically, the expandable cage is shaped and sized to allow the ultrasound transducer to rotate and axially translate back and forth within the expandable cage, such as to generate a three-dimensional image of the tissue. Typically, during rotation of the transluminal ablation catheter which causes rotation of the ultrasound transducer, the expandable cage is held stationary. For some applications, the apparatus comprises a rotational-force reduction mechanism for reducing rotational force applied to the expandable cage during rotation of the transluminal ablation catheter.

Additionally, or alternatively, in accordance with some applications of the present invention, the ultrasound transducer comprises a curved piezoelectric ultrasound transducer shaped to define a convex surface facing outwardly from a longitudinal axis of the transducer. Typically, providing the curved piezoelectric ultrasound transducer widens a thermal profile of the tissue (relative to the piezoelectric ultrasound transducer were uncurved, ceteris paribus) such that a larger portion of the tissue is heated more effectively, thereby facilitating effective and more rapid ablation of the tissue.

There is therefore provided, in accordance with some applications of the present invention, apparatus for use with tissue of a subject, the apparatus including:
a transluminal ablation catheter including:
   at least one ultrasound transducer configured to be inserted into a chamber of the subject's heart, and: (a) to ablate tissue of the subject by applying ultrasound energy to the tissue, and (b) to image tissue of the subject by applying non-ablating ultrasound energy to the tissue; and
   an expandable cage configured to be disposed around the at least one ultrasound transducer,
   the at least one ultrasound transducer being configured to rotate and axially translate back and forth within the expandable cage, such as to generate a three-dimensional image of the tissue.

In some applications, the at least one ultrasound transducer is configured to be inserted into a left atrium in a vicinity of a pulmonary vein ostium and is configured to ablate tissue of the pulmonary vein ostium, to thereby electrically isolate the pulmonary vein.

In some applications, the tissue includes tissue of an ostium of a lumen that extends from the chamber of the subject's heart, and the ultrasound transducer is configured to generate a three-dimensional image of the tissue of the ostium of the lumen by applying non-ablative ultrasound energy to the tissue of the ostium of the lumen.

In some applications, the tissue includes tissue of an ostium of a lumen that extends from the chamber of the subject's heart, and the expandable cage includes a plurality of struts, at least a portion of the struts being curved outwardly at at least two locations along the strut, such that the cage is configured to temporarily anchor a distal portion of the transluminal ablation catheter in the lumen by the portion of the struts contacting a wall of the lumen.

In some applications, the tissue includes tissue of an ostium of a lumen that extends from the chamber of the subject's heart, and the expandable cage has a central portion and a distal portion and the expandable cage is shaped to define a nipple-like structure by the central portion having a diameter that is greater than a diameter of the distal portion such that the distal portion is shaped and sized to be inserted into an ostium of the lumen to temporarily anchor the distal in the lumen by the contacting a wall of the lumen

In some applications, the at least one ultrasound transducer is configured to generate ultrasound energy at a frequency of 8-20 MHz.

In some applications, the at least one ultrasound transducer is shaped to define a convex surface facing outwardly from a longitudinal axis of the transducer, and having a width of 0.5-3 mm and a radius of curvature of 0.75-5 mm.

In some applications, the tissue includes tissue of an ostium of a lumen that extends from the chamber of the subject's heart, and at least a portion of the plurality of struts include electrically conductive struts that are configured to contact tissue of an ostium of the lumen and to ablate tissue of the ostium of the lumen that is in contact with the electrically conductive struts by driving current into the tissue of the ostium of the lumen.

In some applications, at least a portion of the electrically conductive struts include an insulated portion and an electrically conductive portion, and the electrically conductive portion is configured to contact tissue of the ostium of the lumen and to ablate tissue of the ostium of the lumen.

In some applications, at least a portion of plurality of the struts are shaped to define an aperture formed in the strut through which ultrasound energy is transmitted from the ultrasound transducer to the tissue.

In some applications, within the portion of plurality of the struts teach of the struts has a width 0.5-1 mm, and the aperture in the strut has a width of 0.25-0.5 mm. In some applications, a thickness of the strut is 0.1-0.25 mm.

In some applications, the transluminal ablation catheter includes an elongated shaft including a proximal portion including a handle, and a distal portion to which the at least one ultrasound transducer is coupled. In some applications, the elongated shaft is configured to be rotatable, such as to rotate the ultrasound transducer, and the transluminal ablation catheter includes one or more sensors coupled to the distal portion of the elongated shaft and configured to detect a rotational position of the distal portion of the elongated shaft.

In some applications:
the elongated shaft is configured to be rotatable, such as to rotate the ultrasound transducer; and
the transluminal ablation catheter further includes a rotational-force reduction mechanism configured to reduce rotational force applied to the expanded cage by the elongated shaft upon rotation of the elongated shaft, such as to hold the expanded cage stationary during rotation of the ultrasound transducer.

In some applications,
the at least one ultrasound transducer is configured to apply the non-ablating ultrasound energy to the tissue such that at least a portion of the non-ablating ultrasound energy is reflected and received by the ultrasound transducer; and
the apparatus further includes a computer processor configured to assess a parameter of the reflected energy to determine a parameter of the ultrasound energy to be applied by the ultrasound transducer to ablate the tissue; and
the at least one ultrasound transducer is configured to apply the ultrasound energy to the tissue based on the determined parameter.

In some applications, the apparatus further includes an inflatable element configured to be disposed around the ultrasound transducer. In some applications, the inflatable element is configured to be inflated with at least one of water and saline.

In some applications, the at least one ultrasound transducer includes:
a first ultrasound transducer configured to ablate the tissue of the subject by transmitting ablative ultrasound energy toward the tissue; and
a second ultrasound transducer configured to image the tissue of the subject by transmitting one or more pulses of pulse-echo ultrasound energy toward the tissue and receiving a reflection of the transmitted pulse-echo ultrasound energy, and the second ultrasound transducer is configured to rotate and axially translate back and forth within the expandable cage, such as to generate a three-dimensional image of the tissue.

In some applications, the transluminal ablation catheter includes:
a first support configured to support the first ultrasound transducer and enable transmitting of the ablative ultrasound energy toward the tissue; and
a second damping support that is configured to support the second ultrasound transducer and provide a higher level of damping than damping provided by the first support, such as to enable the second ultrasound transducer to receive the reflection of the transmitted pulse-echo ultrasound energy, while the first ultrasound transducer is transmitting the ablative ultrasound energy toward the tissue.

In some applications:
the first support includes an air barrier configured to allow vibration of the first ultrasound transducer during transmitting of the ablative ultrasound energy toward the tissue; and
the second damping support includes a mechanical support including at least one of a backing layer and a damping element.

There is further provided, in accordance with some applications of the present invention, apparatus for use with a lumen of a subject that extends from a chamber of a heart of the subject, the apparatus including:
a transluminal ablation catheter a distal portion of which is configured to be positioned inside the subject's lumen, the transluminal ablation catheter including:
   at least one ultrasound transducer configured to be inserted into the chamber of the subject's heart, and to ablate tissue of an ostium of the lumen by applying ultrasound energy to the tissue of the ostium; and
   an expandable cage including struts and configured to be disposed around the ultrasound transducer, the expandable cage having a central portion and a distal portion and, when disposed in a in a non-radially constrained configuration, being shaped to define a nipple-like structure, such that the central portion has a diameter that is greater than a diameter of the distal portion and the distal portion is shaped and sized to be inserted into the ostium of the lumen to temporarily anchor the distal in the lumen by the contacting a wall of the lumen.

In some applications, the expandable cage is shaped to define the nipple-like structure by the struts defining a convex curvature in the distal portion of the expandable cage, before passing through an inflection point and undergoing a concave curvature within the central portion.

In some applications, the central portion of the expandable cage has a maximum diameter that is up to five times greater than a maximum diameter of the distal portion of the expandable cage.

In some applications, the central portion is configured to remain in the chamber of the heart, when the distal portion is inserted into the ostium of the lumen.

In some applications, the at least one ultrasound transducer is configured to be positioned within a left atrium in a vicinity of a pulmonary vein ostium and is configured to ablate tissue of an ostium of the pulmonary vein, to thereby electrically isolate the pulmonary vein.

In some applications, the expandable cage is rotationally asymmetrical. In some applications, the expandable cage is rotationally symmetrical.

In some applications, the ultrasound transducer includes a side-facing ultrasound transducer. In some applications, the ultrasound transducer includes a distally-facing ultrasound transducer.

In some applications, the ultrasound transducer is further configured to image tissue of the subject by applying non-ablative ultrasound energy to the tissue of the tissue, and the ultrasound transducer is configured to rotate and axially translate back and forth within the expandable cage, such as to generate a three-dimensional image of the tissue.

In some applications, at least a portion of the struts of the expandable cage are shaped to define an aperture formed in the strut through which ultrasound energy is transmitted from the ultrasound transducer to the tissue.

In some applications, within the portion of plurality of the struts teach of the struts has a width 0.5-1 mm, and the aperture in the strut has a width of 0.25-0.5 mm. In some applications, a thickness of the strut is 0.1-0.25 mm.

There is further provided, in accordance with some applications of the present invention, apparatus for use with a lumen of a subject that extends from a chamber of a heart of the subject, the apparatus including:
a transluminal ablation catheter including:
   at least one ultrasound transducer configured to be inserted into the chamber of the subject's heart, and to ablate tissue of an ostium of the lumen by applying ultrasound energy to the tissue of the ostium; and
   an expandable cage configured to be disposed around the ultrasound transducer and including a plurality of struts, at least a portion of the struts shaped to define an aperture formed in the strut through which ultrasound energy is transmitted from the ultrasound transducer to the tissue.

In some applications, each of the struts of the portion of the struts has a width 0.5-1 mm, and the aperture in each of the struts of the portion of the struts has a width of 0.25-0.5 mm. In some applications, at least a portion of the plurality of struts have a thickness of 0.1-0.25 mm.

There is further provided, in accordance with some applications of the present invention, apparatus for use with a lumen of a subject that extends from a chamber of a heart of the subject, the apparatus including:
a transluminal ablation catheter including:
   at least one ultrasound transducer configured to be inserted into the chamber of the subject's heart, and to image tissue of the subject; and
   an expandable cage configured to be disposed around the ultrasound transducer, the expandable cage including a plurality of struts, at least a portion of the struts including electrically conductive struts configured to contact tissue of the ostium of the lumen and to ablate the tissue that is in contact with the electrically conductive struts by driving current into the tissue.

In some applications, at least a portion of the electrically conductive struts include an insulated portion and an electrically conductive portion, and the electrically conductive portion is configured to contact the tissue of the ostium of the lumen and to ablate tissue of the ostium of the lumen.

In some applications, the expandable cage is configured to drive radiofrequency (RF) current into the tissue. In some applications, the expandable cage is configured to drive alternating current (AC) into the tissue. In some applications, the expandable cage is configured to drive direct current (DC) into the tissue.

In some applications, the at least one ultrasound transducer is configured to rotate and axially translate back and forth within the expandable cage, such as to generate a three-dimensional image of the tissue of the ostium of the lumen.

In some applications, the at least one ultrasound transducer is configured to ablate tissue of the ostium of the lumen by applying ultrasound energy to the tissue of the ostium of the lumen.

In some applications, the at least one ultrasound transducer is configured to be inserted into a left atrium in a vicinity of a pulmonary vein ostium and is configured to ablate tissue of an ostium of the pulmonary vein, to thereby electrically isolate the pulmonary vein.

There is further provided, in accordance with some applications of the present invention, apparatus for use with a chamber of a heart of the subject, the apparatus including:
a transluminal ablation catheter including:
   at least one ultrasound transducer configured to be inserted into the chamber of the subject's heart, and to ablate tissue of the subject by applying ultrasound energy to the tissue;
   an inflatable element configured to be disposed around the ultrasound transducer; and
   an expandable cage configured to be disposed around the inflatable element, and to temporarily anchor the transluminal ablation catheter within the chamber of the subject's heart by contacting tissue of the subject.

In some applications, the inflatable element is configured to be inflated with water. In some applications, the inflatable element is configured to be inflated with saline.

In some applications, the at least one ultrasound transducer is configured to generate ultrasound energy at a frequency of 8-20 MHz. In some applications, the at least one ultrasound transducer is configured to generate ultrasound energy at a frequency of 10-12 MHz. In some applications, the at least one ultrasound transducer is configured to generate ultrasound energy at a frequency of 11 MHz.

There is further provided, in accordance with some applications of the present invention, apparatus including:
a transluminal ablation catheter including:
   an ultrasound transducer configured to ablate tissue of a subject by transmitting ultrasound energy toward the tissue; and
   a computer processor configured to:
      detect an indication of blood charring in the vicinity of the ultrasound transducer, and
      inhibit application of ultrasound energy from the ultrasound transducer in response to the detected indication of blood charring.

In some applications:
the ultrasound transducer is further configured to transmit one or more pulses of pulse-echo ultrasound energy toward the tissue and to receive a reflection of the transmitted pulse-echo ultrasound energy; and
the computer processor is configured to detect the indication of blood charring in the vicinity of the ultrasound transducer by determining a parameter of the reflected pulse-echo ultrasound energy.

In some applications, the ultrasound transducer is configured to transmit the ultrasound energy to ablate the tissue at a power level of 3-50 W, and the ultrasound transducer is configured to transmit the pulse-echo ultrasound energy at a power level of less than 2 W.

In some applications:
the ultrasound transducer is a first ultrasound transducer;
the transluminal ablation catheter includes a second ultrasound transducer configured to transmit pulse-echo ultrasound energy to the tissue, and to receive a reflection of the transmitted pulse-echo ultrasound energy; and
the computer processor is configured to detect the indication of blood charring in the vicinity of the ultrasound transducers by determining a parameter of the reflected pulse-echo ultrasound energy.

In some applications, the first ultrasound transducer is configured to transmit the ultrasound energy to ablate the tissue at a power level of 3-50 W, and the second ultrasound transducer is configured to transmit the pulse-echo ultrasound energy at a power level of less than 2 W.

There is further provided, in accordance with some applications of the present invention, apparatus for use with a lumen of a subject that extends from a chamber of a heart of the subject, the apparatus including:
a transluminal ablation catheter a distal portion of which is configured to be positioned inside the subject's lumen, the transluminal ablation catheter including:
   at least one ultrasound transducer configured to be inserted into the chamber of the subject's heart, and to ablate tissue of an ostium of the lumen by applying ultrasound energy to the tissue of the ostium; and
   an expandable cage configured to be disposed around the ultrasound transducer, the expandable cage including a plurality of struts, at least a portion of the struts being curved outwardly at at least two locations along the strut, such that the cage is configured to temporarily anchor the distal portion of the transluminal ablation catheter in the lumen by the portion of the struts contacting a wall of the lumen.

In some applications, the at least one ultrasound transducer is configured to be inserted into a left atrium in a vicinity of a pulmonary vein ostium and is configured to ablate tissue of an ostium of the pulmonary vein, to thereby electrically isolate the pulmonary vein.

In some applications, the expandable cage is rotationally asymmetrical. In some applications, the expandable cage is rotationally symmetrical.

In some applications, the expandable cage is configured, at the curved locations, to adjust a distance between the transducer and the tissue by pushing the tissue by applying pressure when contacting the wall of the lumen.

In some applications, each of the struts that are curved outwardly at at least two locations is shaped such a radius of curvature of a first curved portion is 10-20 mm, and a radius of curvature of a second curved portion is 5-10 mm.

In some applications, at least a portion of the plurality of struts include electrically conductive struts that are configured to contact tissue of the ostium of the lumen and to ablate tissue of the ostium of the lumen that is in contact with the electrically conductive struts by driving current into the tissue of the ostium of the lumen.

In some applications, at least a portion of the electrically conductive struts include an insulated portion and an electrically conductive portion, and the electrically conductive portion is configured to contact tissue of the ostium of the lumen and to ablate tissue of the ostium of the lumen.

In some applications, the expandable cage is configured to drive radiofrequency (RF) current into the tissue. In some applications, the expandable cage is configured to drive alternating current (AC) into the tissue. In some applications, the expandable cage is configured to drive direct current (DC) into the tissue.

In some applications, at least a portion of plurality of the struts are shaped to define an aperture formed in the strut through which ultrasound energy is transmitted from the ultrasound transducer to the tissue.

In some applications, within the portion of plurality of the struts teach of the struts has a width 0.5-1 mm, and the aperture in the strut has a width of 0.25-0.5 mm. In some applications, a thickness of the strut is 0.1-0.25 mm.

In some applications, the transluminal ablation catheter includes an elongated shaft including a proximal portion including a handle, and a distal portion to which the at least one ultrasound transducer is coupled. In some applications, the apparatus further includes one or more sensors coupled to the distal portion of the elongated shaft and configured to detect a rotational position of the distal portion of the elongated shaft.

In some applications, the ultrasound transducer includes a side-facing ultrasound transducer. In some applications, the ultrasound transducer includes a distally-facing ultrasound transducer.

In some applications, a distal tip of transluminal ablation catheter includes an electrode configured to ablate tissue of a wall of the lumen.

In some applications, the ultrasound transducer is further configured to image tissue of the subject by applying non-ablative ultrasound energy to the tissue, and the ultrasound transducer is configured to rotate and axially translate back and forth within the expandable cage, such as to generate a three-dimensional image of the tissue.

In some applications, the tissue includes tissue of the ostium of the lumen and the ultrasound transducer is configured to image tissue of the ostium of the lumen by applying non-ablative ultrasound energy to the tissue of the ostium of the lumen.

In some applications, the apparatus further includes one or more acoustic fiducial markers disposed on the expandable cage.

There is further provided, in accordance with some applications of the present invention, apparatus including:
a transluminal ablation catheter including:
a piezoelectric ultrasound transducer configured to be inserted into the chamber of the subject's heart the piezoelectric ultrasound transducer being shaped to define a convex surface facing outwardly from a longitudinal axis of the transducer, and having a width of 0.5-3 mm and a radius of curvature of 0.75-5 mm.

In some applications, the at least one ultrasound transducer is configured to be positioned within a left atrium in a vicinity of a pulmonary vein ostium and is configured to ablate tissue of the pulmonary vein ostium, to thereby electrically isolate the pulmonary vein.

In some applications, the ultrasound transducer has a width of 1-2 mm. In some applications, the ultrasound transducer has a thickness of 0.1-0.3 mm. In some applications, the ultrasound transducer has a length of 2-20 mm.

There is further provided, in accordance with some applications of the present invention, apparatus for use with a lumen of a subject that extends from a chamber of a heart of the subject, the apparatus including:
a transluminal ablation catheter including:
   an ultrasound transducer configured to tissue of the subject by transmitting ultrasound energy to the tissue;
   a sensor in operable communication with the transluminal ablation catheter and configured to detect a change in blood flow in the lumen; and
   a computer processor configured to:
      determine an optimal tissue target site for ablation based on the detected changes in blood flow, and
      drive the ultrasound transducer to transmit the ultrasound energy to the optimal tissue target.

In some applications, the sensor includes a Doppler sonography device. In some applications, the sensor is configured to detect a sound indication being indicative of the change in blood flow in the lumen.

In some applications, the transluminal ablation catheter further includes an actuator configured to adjust a position of the ultrasound transducer in response to the detected change in flow, so that the transmitted ultrasound energy is applied to the optimal tissue target site.

In some applications:
the optimal tissue target includes an ostium of the lumen,
the sensor is configured to detect a change in blood flow between the lumen and the chamber of the heart, and
the computer processor is configured to drive the ultrasound transducer to transmit the ultrasound energy to the ostium of the lumen based on the detected change in flow between the lumen and the chamber of the heart.

In some applications:
the optimal tissue target includes a pulmonary vein ostium,
the ultrasound transducer is configured to be positioned within an atrium of a heart of the subject,
the sensor is configured to detect a change in blood flow between a pulmonary vein and the atrium, and
the computer processor is configured to drive the ultrasound transducer to transmit the ultrasound energy to the pulmonary vein ostium based on the detected change in flow between the pulmonary vein and the atrium.

There is further provided, in accordance with some applications of the present invention, a method including:
advancing into an atrium of a heart of a subject a transluminal ablation catheter that includes at least one ultrasound transducer;
using a sensor, detecting a change in a flow of blood in the vicinity of the at least one ultrasound transducer to determine a location of a pulmonary vein with respect to the atrium; and
activating the ultrasound transducer to ablate tissue at a pulmonary vein ostium in response to determining the location.

In some applications, using the sensor includes using Doppler sonography. In some applications, using the sensor includes using the sensor to detect a sound indication indicative of the change in the flow of blood. In some applications, the method further includes adjusting a position of the ultrasound transducer in response to determining the location.

In some applications, the method further includes:
generating three-dimensional image data of the atrium using the ultrasound transducer, and
using the image data in combination with the change in flow detected by the sensor to determine the location of the pulmonary vein with respect to the atrium.

There is further provided, in accordance with some applications of the present invention, a method including:
advancing at least one ultrasound transducer into a heart chamber of a subject to ablate myocardial tissue of the subject;
applying non-ablating ultrasound energy to the myocardial tissue, such that at least a portion of the non-ablating ultrasound energy is reflected and received by the ultrasound transducer;
assessing a parameter of the reflected energy to determine a parameter of the ultrasound energy to be applied by the ultrasound transducer to ablate the myocardial tissue; and
applying ultrasound energy to the myocardial tissue based on the determined parameter.

In some applications, assessing a parameter of the reflected energy includes determining an energy level of the reflected energy. In some applications, assessing a parameter of the reflected energy to determine a parameter of the ultrasound energy to be applied by the ultrasound transducer to ablate the myocardial tissue includes assessing a parameter of the reflected energy to determine a power level of the ultrasound energy to be applied by the ultrasound transducer to ablate the myocardial tissue. In some applications, assessing a parameter of the reflected energy further includes assessing a parameter of the reflected energy to determine whether the ultrasound transducer is in a desired location and adjusting a position of the ultrasound transducer in response thereto.

There is further provided, in accordance with some applications of the present invention, apparatus for use with a lumen of a subject that extends from a chamber of a heart of the subject, the apparatus including:
a transluminal ablation catheter a including:
   an elongated shaft having proximal and distal portions;
   at least one ultrasound transducer coupled to the distal portion of the shaft and that is configured to be inserted into the chamber of the subject's heart, and to ablate tissue of an ostium of the lumen by applying ultrasound energy to the tissue, the elongated shaft being configured to be rotatable, such as to rotate the ultrasound transducer;
   an expandable cage disposed at the distal portion of the shaft and configured to surround the ultrasound transducer in an expanded state of the cage when the ultrasound transducer is positioned within the chamber of the subject's heart; and
   a rotational-force reduction mechanism configured to reduce rotational force applied to the expanded cage by the elongated shaft upon rotation of the elongated shaft, such as to hold the expanded cage stationary during rotation of the ultrasound transducer.

There is further provided, in accordance with some applications of the present invention, apparatus including:
a transluminal ablation catheter including:
   a first ultrasound transducer configured to ablate tissue of a subject by transmitting ablative ultrasound energy toward the tissue; and
   a second ultrasound transducer configured to transmit one or more pulses of pulse-echo ultrasound energy toward the tissue and to receive a reflection of the transmitted pulse-echo ultrasound energy;
   a first support configured to support the first ultrasound transducer and configured to enable transmitting of the ablative ultrasound energy toward the tissue; and
   a second support that is configured to support the second ultrasound transducer and provide a higher level of damping than damping provided by the first support, such as to enable the second transducer to receive the reflection of the transmitted pulse-echo ultrasound energy, while the first ultrasound transducer is transmitting the ablative ultrasound energy toward the tissue.

In some applications,
the first support includes an air barrier configured to allow vibration of the first ultrasound transducer during transmitting of the ablative ultrasound energy toward the tissue; and
the second support includes a mechanical support including at least one of a backing layer and a damping element.

The present invention will be more fully understood from the following detailed description of embodiments thereof, taken together with the drawings, in which:

### BRIEF DESCRIPTION OF THE DRAWINGS

FIGS. 1A, 1B and 1C are schematic illustrations of an apparatus for application of ultrasound energy to tissue within a body of a subject, in accordance with some applications of the present invention;

FIG. 1D is a schematic illustration of an expandable cage of the apparatus for application of ultrasound energy to tissue within a body of a subject, in accordance with some applications of the present invention;

FIG. 1E is a schematic illustration of the apparatus for application of ultrasound energy to tissue positioned within a body of a subject, in accordance with some applications of the present invention;

FIG. 2A is a schematic illustration of apparatus for application of ultrasound energy to tissue within a body of a subject, in accordance with some applications of the present invention;

FIG. 2B is a graph showing the effect of the distance of an ultrasound transducer from an ablation site on absorption of the ultrasound energy by various mediums through which the ultrasound energy is transmitted in accordance with some applications of the present invention;

FIG. 3A is a schematic illustration of apparatus for application of ultrasound energy to tissue within a body of a subject, in accordance with some applications of the present invention;

FIGS. 3B and 3C are pictures of components of the apparatus for application of ultrasound energy to tissue within a body of a subject, in accordance with some applications of the present invention;

FIGS. 4A, 4B, 4C, 4D, 4E, and 4F are schematic illustrations of a rotational-force reduction mechanism for reducing rotational force applied to the expandable cage during rotation of the ultrasound transducer, in accordance with some applications of the present invention;

FIG. 5A is a schematic illustration of a curved piezoelectric ultrasound transducer, in accordance with some applications of the present invention;

FIGS. 5B and 5C are images of an ultrasound energy transmission profile using the ultrasound transducer of FIG. 5A, in accordance with some applications of the present invention;

FIG. 5D is a graph showing the effect of various dimensions of the ultrasound transducer of FIG. 5A on an angle of emission of the transducer, in accordance with some applications of the present invention; and

FIG. 6 is a flowchart showing steps of a method that is performed, in accordance with some applications of the present invention.

### DETAILED DESCRIPTION OF EMBODIMENTS

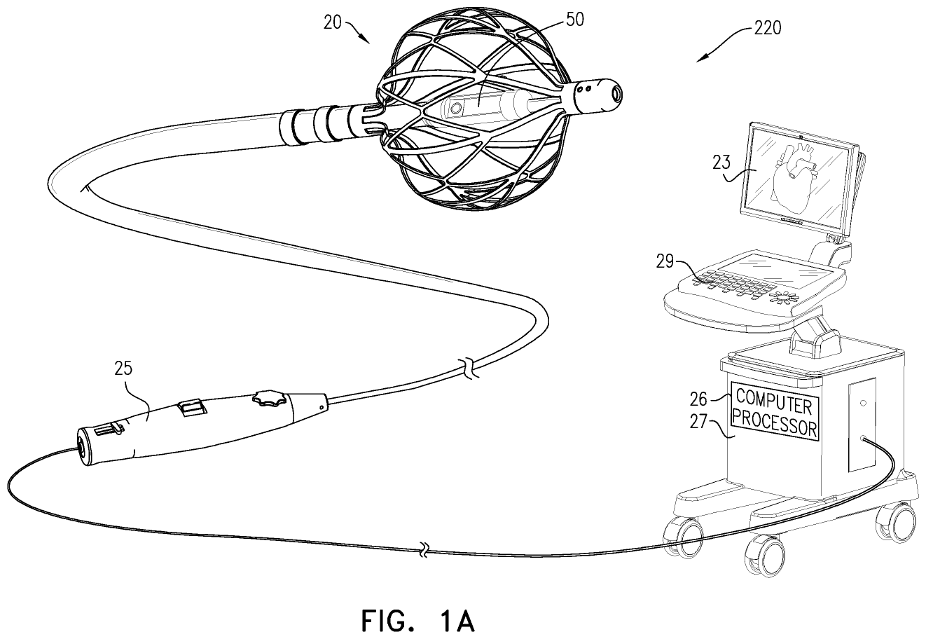

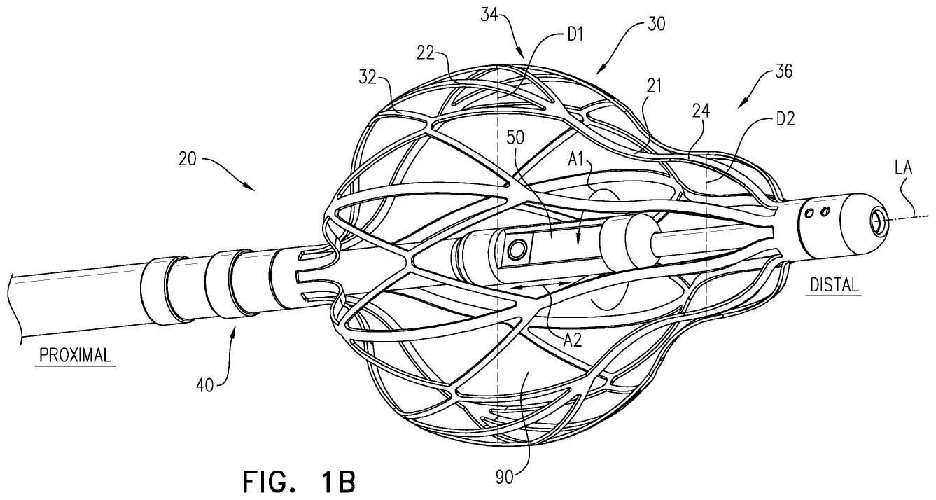

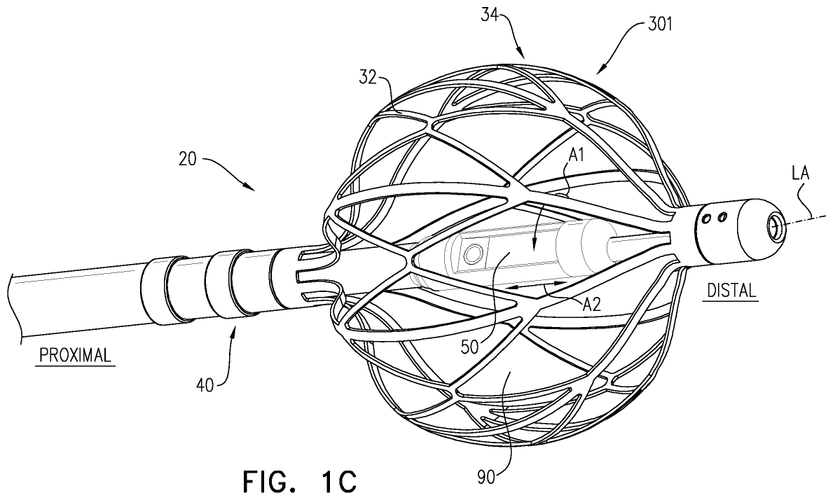

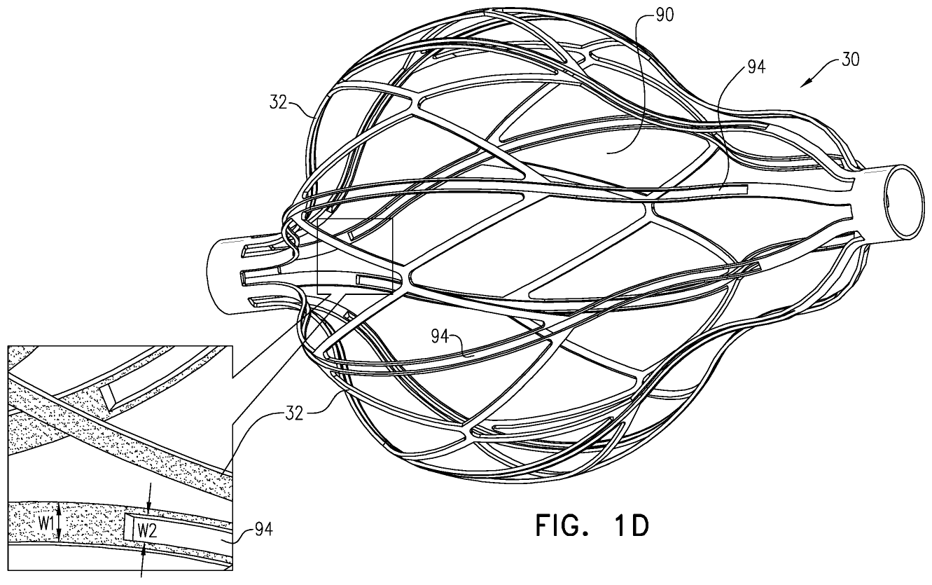

Reference is made to FIGS. 1A, 1B, and 1C, which are schematic illustrations of an apparatus for application of ultrasound energy to tissue within a body of a subject, in accordance with some applications of the present invention.

FIG. 1A shows an overview of system 220 for ultrasound tissue treatment, including a control console 27, a handle 25, and apparatus 20 for application of the ultrasound energy to the tissue within the body of the subject. FIGS. 1B-C show embodiments of the apparatus 20 having different shaped expandable cages as will be described in further detail hereinbelow.

System 220 shown in FIG. 1A, typically includes apparatus 20, which comprises ultrasound transducer 50 (and/or ultrasound transducers 52/150 described hereinbelow) configured to apply ultrasound energy toward a target tissue. Apparatus 20 is typically operatable with control console 27, which includes a computer processor 26 and a display 23.

For example, computer processor 26 is configured to detect various parameters related to application of the ultrasound energy (such as parameters of applied and/or reflected ultrasound energy, etc.), and drive the ultrasound transducer to transmit the ultrasound energy (e.g., by selecting optimal ultrasound energy application parameters, etc.), as will be described in further detail hereinbelow. For some applications, computer processor 26 drives the ultrasound transducer to rotate and/or translate back and forth, as will be described hereinbelow. For example, the computer processor may control motion of the ultrasound transducer through an actuator (e.g., a motor) housed in handle 25 (or elsewhere system 220). For some applications, computer processor 26 is configured to control apparatus 20 in response to user input received through handle 25 or other user input interfaces (such as keyboard 29)

Computer processor 26 is typically a hardware device programmed with computer program instructions to produce a special-purpose computer. For example, when programmed to perform the techniques described herein, computer processor 26 typically acts as a special-purpose, ultrasound energy application computer processor.

Reference is now made to FIG. 1B, which is a schematic illustration of apparatus 20 for application of ultrasound energy to tissue of a target anatomical structure within a body of a subject, in accordance with some applications of the present invention. Apparatus 20 is typically configured for use with a lumen of a subject that extends from a chamber of a heart of the subject. For some applications, the chamber of the heart is an atrium of the heart and the lumen extending from the atrium is a pulmonary vein.

Typically, apparatus 20 applies the ultrasound energy to treat cardiac arrhythmias, such as atrial fibrillation. In accordance with some applications of the present invention, the ultrasound energy is applied towards myocardial tissue, and in particular towards sites within myocardial tissue which are involved in triggering, maintaining, or propagating cardiac arrhythmias, e.g., pulmonary vein ostia. As such, as described hereinabove, apparatus 20 is shaped and sized for use with the pulmonary vein that extends from the left atrium of the heart. Apparatus 20 is configured to apply the ultrasound energy to cause ablation of the tissue of the pulmonary vein ostia resulting in scarring of the tissue at the ablated sites. The scars typically block abnormal electrical pulses generated in the pulmonary vein ostia from propagating into the heart chambers, thereby electrically isolating the pulmonary veins from the atrium and reducing or preventing cardiac arrhythmias.

For some applications, apparatus 20 comprises a transluminal ablation catheter 40 comprising an elongated shaft having a proximal portion comprising handle 25 (e.g., shown in FIG. 1A), and a distal portion to which at least one ultrasound transducer 50 is coupled. It is noted that in this context, in the specification and in the claims, “proximal” means closer to the user of the apparatus, and “distal” means farther from the user, and farther into the subject's body from the orifice through which the apparatus is originally placed into the body.

Transluminal ablation catheter 40 facilitates advancement of ultrasound transducer 50 into the chamber of the heart of the subject, (e.g., the atrium), in a minimally invasive procedure. Ultrasound transducer 50 is typically inserted into the atrium to ablate tissue of the ostium of the lumen, e.g., the pulmonary vein ostium, by applying ultrasound energy to the tissue of the ostium. Additionally, or alternatively to applying ablative ultrasound energy, ultrasound transducer 50 is configured to image tissue of the subject by applying non-ablative ultrasound energy.

For some applications, ultrasound transducer 50 comprises a side-facing ultrasound transducer. For other applications, ultrasound transducer 50 comprises a distally-facing ultrasound transducer. For some applications, more than one ultrasound transducer 50 is coupled to transluminal ablation catheter 40, with at least one distally-facing ultrasound transducer and at least one side-facing ultrasound transducer. Additionally, for some applications, a distal tip of transluminal ablation catheter 40 comprises an electrode configured to drive current into the tissue designated for ablation treatment to further assist in lesion formation in the tissue.

For some applications, apparatus 20 further comprises an anchoring element, e.g., an expandable cage, which is shaped to define a three-dimensional structure configured to be disposed around ultrasound transducer 50 at the distal portion of transluminal ablation catheter 40. FIG. 1B shows an expandable cage 30, which is shaped in accordance with some applications of the present invention, whereas FIGS. 1A and 1C show an expandable cage 301 shaped in accordance with other applications of the present invention.

As shown in FIG. 1B, expandable cage 30 comprises a plurality of struts 32 (e.g., flexible struts which may be made from an elastic metal, such as, nitinol, stainless steel, nickel-titanium, or a combination thereof). As noted above, in some cases, the struts are referred to herein as “flexible wires,” with these two terms being used interchangeably throughout the specification and the claims. Expandable cage 30 is configured to position the distal portion of transluminal ablation catheter 40 within the lumen of the subject that extends from a heart chamber of the subject, e.g., within the lumen of pulmonary veins that extend from the left atrium of the heart.

Expandable cage 30 positions and temporarily anchors the distal portion of transluminal ablation catheter 40 in the lumen by struts 32 contacting a wall of the lumen. Typically, expandable cage 30 engages walls of the lumen, e.g., the pulmonary vein, without blocking blood flow through the lumen (as shown, expandable cage 30 is shaped so as to provide passage of blood therethrough).

Expandable cage 30 is typically delivered via transluminal ablation catheter 40 to the target anatomical structure site within the body of the subject in a collapsed state thereof. Struts 32 typically include a shape memory alloy that expands automatically from a collapsed configuration to an expanded configuration upon deployment in the lumen, e.g., in the pulmonary vein. When withdrawing transluminal ablation catheter 40 from the subject's body is desired, struts 32 are collapsed from the expanded state into a predefine collapsed shape by application of mechanical force of compression or tension in the handle. Shapes of the expandable cage and portions thereof that are described herein typically refer to the shape of the expandable cage which the expandable cage is shape set to assume when the expandable cage is in a non-radially constrained configuration (e.g., when deployed at the target anatomical structure site).

For some applications, expandable cage 30 is rotationally symmetrical. For other applications, expandable cage 30 is rotationally asymmetrical.

For some applications, expandable cage 30 is configured to position transducer 50 and/or catheter 40 in the center of the lumen. For other applications, expandable cage 30 is configured to position transducer 50 and/or catheter 40 asymmetrically within the lumen, i.e., not in the center of the lumen of the blood vessel. For example, expandable cage 30 may position transducer 50 so as to aim at a portion of the lumen wall designated for ablation and/or imaging. For example, the expandable cage 30 may be configured to anchor transluminal ablation catheter 40 such that ultrasound transducer 50 transmits the ultrasound energy towards the ostium of the lumen when the ostium is designated for ablation and/or imaging. For some applications, expandable cage 30 is configured to maintain a radial separation between ultrasound transducer 50 and the wall of the lumen and to position transducer 50 at a desired distance from the site designated for imaging or ablation. For example, expandable cage 30 is configured to adjust a distance between transducer 50 and the tissue designated for ablation and/or imaging by pushing the tissue by applying pressure when contacting the wall of the lumen. For some applications, expandable cage 30 is configured to maintained ultrasound transducer 50 at a fixed distance from the tissue during imaging to detect lesion formation while such that artifacts associated with tissue movement, are reduced.

For some applications, expandable cage 30 has a nipple-like structure by being shaped such that a distal portion 36 of expandable cage 30 is narrower than a central portion 34 of expandable cage 30. Having a narrower distal portion 36 typically facilitates insertion of expandable cage 30 into relatively small or narrow anatomical structures of various diameters, e.g., the lumen of blood vessels such as the pulmonary vein. For example, for some applications, only narrow distal portion 36 of cage 30 is inserted into the pulmonary vein, while the rest of apparatus 20 remains in the atrium. For some applications, the narrow distal portion 36 of cage 30 thereby anchors cage 30 with respect to the pulmonary vein.

Typically, starting at the distal end of expandable cage 30, the struts first define a convex curvature (with respect to the outside of expandable cage) before passing through an inflection point 21 and undergoing a concave curvature. The narrow distal portion extends from the distal end of the expandable cage until the inflection point.

For some applications, a maximum diameter D1 of expandable cage 30 at central portion 34 is up to five times greater than a maximum diameter D2 of expandable cage 30 at narrow distal portion 36, thereby facilitating insertion of narrow distal portion 36 into the pulmonary vein.

For some applications, expandable cage 30 is structured such that at least a portion of struts 32 are curved outwardly (i.e., convexly curved with respect to the outside of expandable cage) at at least two locations along a single strut, e.g., at curved locations 22 and 24. For some application, the outwardly curved areas anchor transluminal ablation catheter 40 in the lumen by contacting a wall of the lumen. For some applications, struts 32 that are curved outwardly at at least two locations (e.g., 22 and 24) are shaped such that a radius of curvature of first curved location 22 is 10-20 mm, and a radius of curvature of second curved location 24 is 5-10 mm. It is noted that the radius of curvature of curved locations 22 and 24 is such that when these portions are urged against the wall of the lumen, the tissue is generally not penetrated or injured.

As described hereinabove, for some applications, ultrasound transducer 50 is configured to generate an image of the tissue by applying non-ablative ultrasound energy to the tissue. For some applications, ultrasound transducer 50 generates a three-dimensional image (e.g., of the pulmonary vein ostium) by rotating around a longitudinal axis LA of transluminal ablation catheter 40, in the direction indicated by arrow A1, and longitudinally translating back and forth along longitudinal axis LA in the direction indicated by arrow A2. In accordance with some applications of the present invention, dimensions of expandable cage 30 are such that transducer 50 is allowed to rotate and translate back and forth within the cage in the directions indicated by arrows A1 and A2, while expandable cage 30 is disposed around the transducer.

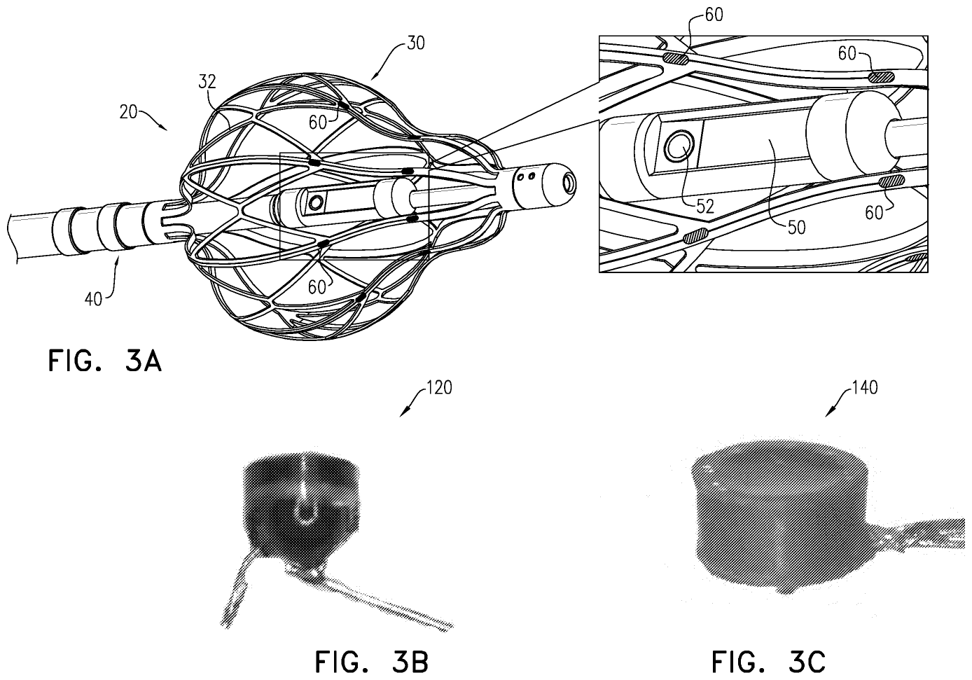

For some applications, expandable cage 30 is additionally configured to electrically stimulate and/or sense electrical signals from the anatomical structure in which apparatus 20 is positioned. For some such applications, expandable cage 30 comprises one or more electrodes 60 (shown in FIG. 3A), configured to record electrical activity prior to, during and/or subsequently to, ablation in order to monitor the ablation procedure and lesion formation. For some applications, ablation treatment parameters (e.g., duration of energy application and/or the level of energy applied to the tissue) are regulated based on the monitoring by one or more electrodes 60.

Additionally, or alternatively, at least a portion of the plurality of struts 32 forming expandable cage 30 comprise electrically conductive struts, e.g., metallic flexible wires, configured to contact tissue of the wall of the lumen (e.g., the ostium of the lumen) and to ablate the tissue that is in contact with the electrically conductive struts/wires by driving current into the tissue. Typically, for such applications, ultrasound transducer 50 is used primarily for imaging the tissue.

For some such applications, at least a portion of the electrically conductive struts comprise an electrically conductive portion and an electrically insulated portion. Typically, in the insulated portion of the strut, the strut is coated with an insulating material, whereas in the electrically conductive portion the strut is exposed. Typically, locations of strut 32 that contact tissue are electrically conductive such that current is driven into the tissue to ablate the tissue (e.g., curved locations 22 and 24 shown in FIG. 1B, that are typically urged against the wall of the lumen).

In accordance with some applications of the present invention, expandable cage 30 drives radiofrequency (RF) current into the tissue through the electrically conductive portions of struts 32. Additionally, or alternatively expandable cage 30 drives alternating current and/or direct current (DC) into the tissue through the electrically conductive portions of struts 32.

Reference is now made to FIG. 1C, which is a schematic illustration of apparatus 20, in accordance with some applications of the present invention. As shown in FIG. 1C, for some applications, apparatus 20 comprises an expandable cage 301 in which struts 32 are shaped to define a spherical shape of expandable cage 301. Expandable cage 301 generally lacks the narrowing of distal portion 36, which provides the nipple-shape of expandable cage 30 (as shown in FIG. 1B). With the exception of narrow distal portion 36 that extends from the distal end of the expandable cage until inflection point 21 (which is absent in expandable cage 301), expandable cage 301 is generally the same as expandable cage 30.

Referring again to FIG. 1A, it is noted that the shape of expandable cage shown in FIG. 1A, is shown by way of illustration and not limitation. It is noted that any other suitable shape of the expandable cage may be used including an expandable cage 30 having a shape as shown in FIG. 1B, an ellipsoidal expandable cage (not shown), or an expandable cage having any other shape, mutatis mutandis. It is further noted that the additional embodiments of the present invention described herein with reference to FIGS. 1D-6 are applicable to either shape of expandable cage 30 or expandable cage 301.

Reference is now made to FIG. 1D, which is a schematic illustration of expandable cage 30 in accordance with some applications of the present invention. As shown in FIG. 1B, struts 32 are structured to form expandable cage 30 such that expandable cage 30 is shaped to define a plurality of relatively large gaps 90 through which the ultrasound energy is transmitted from ultrasound transducer 50 to the tissue designated for ablation and/or imaging. Additionally, for some applications, at least a portion of struts 32 of expandable cage 30 are shaped to define one or more apertures 94 formed in the strut, in order to further facilitate transmission to the ultrasound energy to the tissue (apertures 94 are shown in FIG. 1D). The apertures formed in struts 32 typically increase the area through which the ultrasound energy is transmitted through expandable cage 30, thereby allowing for more ultrasound energy to reach the target tissue, resulting in increased heating of the tissue, leading to more effective ablation of the tissue.

Struts 32 typically have a width that is both sufficiently narrow such as to allow for formation of gaps 90 between struts 32, and also sufficiently wide to provide the required anchoring and stabilization of the distal portion of transluminal ablation catheter 40 in the lumen. Therefore, providing apertures 94 in struts 32 enhances transmission of the ultrasound energy to the tissue through expandable cage 30, while still providing sufficient anchoring of catheter 40 in the lumen. Typically, a width W1 of a single strut 32 is between 0.5-1 mm, e.g., 0.7 mm, and a width W2 of the aperture in the strut is between 0.25-0.5 mm, e.g., 0.35 mm. It is noted that, for some applications, aperture 94 extends along an entire length of strut 32. Alternatively, for some applications, aperture 94 extends along a portion of the length of strut 32.

Reference is still made to FIGS. 1B-D. Typically, a thickness of each one or a portion of the plurality of struts 32 is less than a value of the ultrasound wavelength for the ultrasound frequency generated by ultrasound transducer 50, in accordance with some applications of the present invention. When thickness of strut 32 is smaller than the value of the ultrasound wavelength (when propagating in blood) for the frequency used, interference to the propagation of the ultrasound wave (e.g., obstruction by the strut) is reduced, thereby resulting in more energy reaching the tissue.

In accordance with some applications of the present invention, in order to achieve effective heating and ablation of the tissue, the ultrasound energy is applied at a frequency of 8-20 MHz, e.g., 10-12 MHz, e.g., 11 MHz. Since, generally, ultrasound wavelength decreases with increasing frequency, struts 32 generally have a thickness that is 0.1-0.25 mm (e.g., 0.14-0.2), in order to facilitate propagation of the ultrasound waves through cage 30 when ultrasound transducer 50 is operated, for example, at frequencies of 8-20 MHz, e.g., 10-12 MHz, e.g., 11 MHz.

Reference is now made to FIG. 1E, which is a schematic illustration of apparatus 20 positioned within a body of a subject, in accordance with some applications of the present invention.

For some applications, apparatus 20 is advanced into a left atrium 190, and placed in a location adjacent to, or within, the pulmonary vein ostia. For some applications, a transseptal approach is used to advance apparatus 20 into left atrium 190 (as shown by way of illustration in FIG. 1E). Alternatively, apparatus 20 may be advanced to left atrium 190 using a transapical approach, via the apex of the left ventricle and the mitral valve (approach not shown). Further alternatively, apparatus 20 may be advanced to left atrium 190 via the aorta, the left ventricle, and the mitral valve (approach not shown).

Typically, apparatus 20 is advanced into left atrium 190 with expandable cage 30 in a collapsed state thereof. Struts 32 expand from the collapsed configuration into an expanded configuration within the left atrium of the heart, brining apparatus 20 into an operative state. Apparatus 20 is located adjacent to an ostium of pulmonary vein 160 and to tissue of atrial wall 170, such that a portion of cage 30 (typically a portion of central portion 34) is anchored against wall 170. Distal portion 36 of cage 30 is typically advanced into pulmonary vein 160 in order optimally locate ultrasound transducer 50 with respect to the tissue designated for ablation (typically the pulmonary vein ostia). Additionally, or alternatively, distal portion 36 of expandable cage 30 is advanced into pulmonary vein 160 in order to anchor and maintain apparatus 20 in place during application of the ultrasound energy, by and applying pressure to the walls of the pulmonary vein.

Although FIG. 1E shows ultrasound transducer 50 located outside the pulmonary vein, it is noted that apparatus 20 may be configured such that the ultrasound transducer located further distally along catheter 40 such that it is advanced into the pulmonary vein when distal portion 36 of cage 30 is advanced into the pulmonary vein.

It is further noted that the scope of the present invention includes using the apparatus and methods described herein in anatomical locations other than the left atrium and the pulmonary vein.

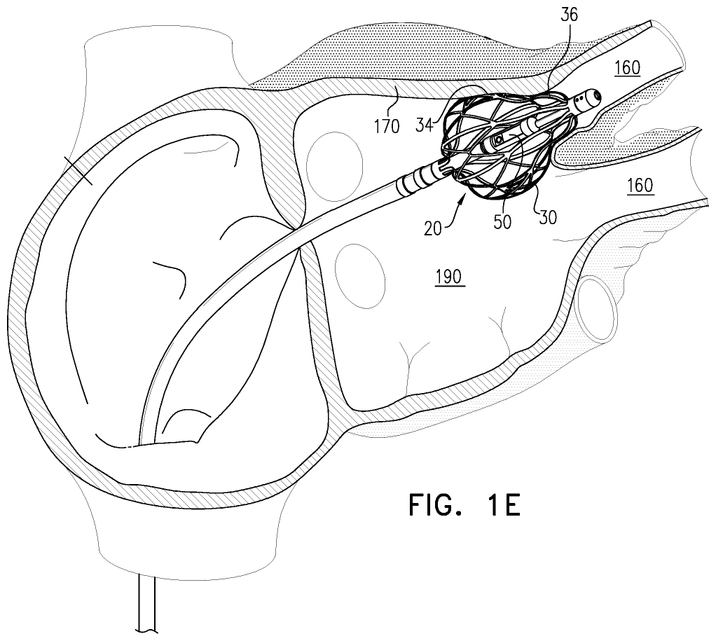

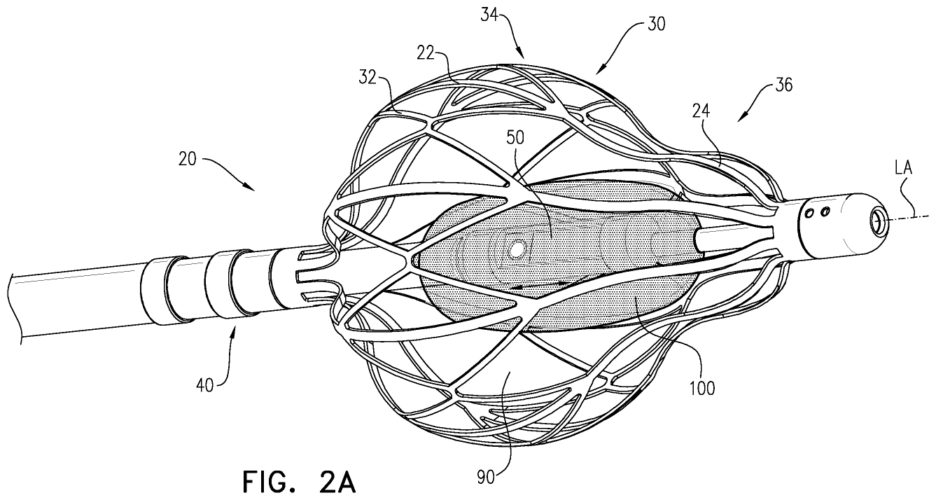

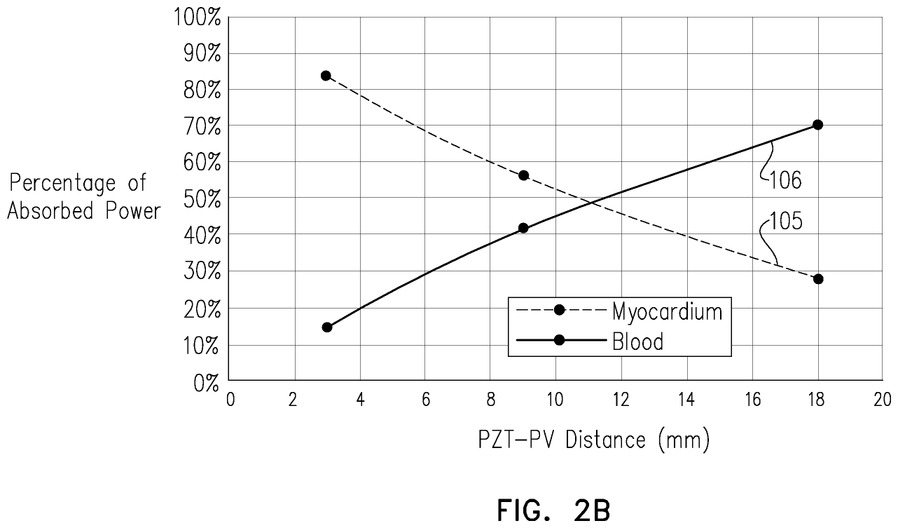

Reference is now made to FIG. 2A, which is a schematic illustration of apparatus 20 for application of ultrasound energy to tissue within a body of a subject, in accordance with some applications of the present invention. Reference is also made to FIG. 2B, which is a graph showing absorption of ultrasound energy by blood (indicated by line 106), and myocardial tissue (indicated by line 105) at increasing distances (in millimeters) of the ultrasound transducer from the tissue site designated for ablation (e.g., the pulmonary vein), typically when a relatively a high frequency of the ultrasound energy is applied (e.g., between 8-20 MHz, e.g., between 10-12 MHz, e.g., 11 MHz, e.g., 11.2 MHz).

As shown in FIG. 2A, for some applications, in addition to expandable cage 30, apparatus 20 comprises a fluid-filled inflatable element 100, e.g., a balloon, configured to be disposed around ultrasound transducer 50. Typically, inflatable element 100 is inflated with a fluid, such as water (e.g., distilled water) and/or saline, and/or any liquid with an acoustic attenuation coefficient similar to saline, through which ultrasound energy is transmitted, but in which absorption of ultrasound energy is negligible. For such applications, expandable cage 30 is disposed around inflatable element 100 to position the distal portion of the transluminal ablation catheter 40 in the lumen, as described hereinabove.

Typically, in the presence of expandable cage 30, but in the absence of inflatable element 100 (such as shown in FIGS. 1B-C), blood is the primary medium through which the ultrasound energy emitted from ultrasound transducer 50 is transmitted to the tissue. This is due to expandable cage 30 shaped to allow blood flow through the lumen of the blood vessel (e.g., the pulmonary vein). When the ultrasound energy from transducer 50 is transmitted through the blood to the tissue, a portion of the energy is absorbed by the blood, resulting in a reduced amount of ultrasound energy reaching the tissue. This is particularly the case when using relatively high frequency ultrasound to cause effective ablation of tissue (e.g., 8-20 MHz, e.g., 10-12 MHz, e.g., 11 MHz), in accordance with some applications of the present invention, compared to use of lower frequencies such as 6 MHz.

Typically, as shown in FIG. 2B, the greater the distance between the piezoelectric element (PZT) of ultrasound transducer 50 and the tissue designated for ablation/imaging (the pulmonary vein (PV)), the more ultrasound energy is absorbed by the blood in the vessel (thus less energy reaching the tissue). For example, when ultrasound transducer 50 is positioned at a distance of approximately 10 mm from the tissue designated for ablation, only a partial amount (e.g., approximately 50%) of the transmitted ultrasound energy reaches the tissue. In such cases it may be the case that the tissue is not sufficiently heated to achieve ablation thereof.

Typically, as shown in FIG. 2B, absorption of the ultrasound energy by the blood (line 106) increases as the distance of the ultrasound waves travel increases and correspondingly absorption of the ultrasound energy by the myocardial tissue decreases (line 105).

Placing fluid-filled (e.g., water-filled) inflatable element 100 around ultrasound transducer 50, as shown in FIG. 2A, partially replaces the blood medium between ultrasound transducer 50 and the tissue designated for ablation. As described hereinabove (and shown in FIG. 2B), ultrasound energy is transmitted through water but substantially not absorbed by it, thereby allowing for a greater amount of the transmitted ultrasound energy to reach the tissue in comparison to if the transmitted ultrasound energy were transmitted through only blood.

For example, inflatable element 100 is inflated with water such that it occupies up to 50% (e.g., 30-40%) of the distance between transducer 50 and the tissue designated for ablation, thereby reducing the amount of ultrasound energy that may have been absorbed by blood in the lumen in the absence of inflatable element 100. Providing inflatable element 100 typically allows operation of ultrasound transducer 50 at lower power levels and for a shorter duration of time in comparison to if the transmitted ultrasound energy were transmitted through only blood.

Advantageously, since inflatable element 100 is used in addition to expandable cage 30 (and therefore not relied on for positioning of ablation catheter 40 in the lumen), inflatable element 100 may be inflated to any desired degree of inflation, for example, based on the anatomical site into which ultrasound transducer is inserted, and/or operation parameters of ultrasound transducer 50. For some applications, inflatable element 100 is inflated with water to a diameter of 6-9 mm, e.g., 8 mm.

Reference is now made to FIG. 3A, which is a schematic illustration of apparatus 20 for application of ultrasound energy to tissue within the body of the subject, in accordance with some applications of the present invention. Reference is also made to FIGS. 3B and 3C, which are pictures of additional components of apparatus 20, in accordance with some applications of the present invention.

In accordance with some applications of the present invention, in addition to applying ultrasound energy for ablation purposes, ultrasound transducer 50 is configured to perform acoustic sensing by transmitting one or more pulses of pulse-echo ultrasound energy towards the designated tissue site and receiving a reflection of the transmitted pulse-echo ultrasound energy. Generally, a parameter of the reflected pulse-echo ultrasound energy can be indicative of the ultrasound energy applied to the tissue and of an effect of the ultrasound energy on the tissue. As described hereinabove, for some applications, apparatus 20 comprises control console 27 and computer processor 26 (shown for example in FIG. 1A), configured to determine the parameter of the reflected pulse-echo ultrasound energy to detect an effect or an indication of the applied ultrasound energy.

For some applications, in addition to ultrasound transducer 50, a second ultrasound transducer 52 is coupled to transluminal ablation catheter 40. For some such applications, first ultrasound transducer 50 is configured to ablate tissue of the lumen by transmitting ablative ultrasound energy toward the tissue, and second ultrasound transducer 52 is configured to transmit pulse-echo ultrasound to the tissue, and to receive a reflection of the transmitted pulse-echo ultrasound. For example, first ultrasound transducer 50 is configured to transmit the ultrasound energy to ablate the tissue at a power level of more than 3 W, e.g., 3 W-50 W, e.g., 6-35 W, and second ultrasound transducer 52 is configured to transmit the pulse-echo ultrasound energy at a power level of up to (e.g., less than) 2 W.

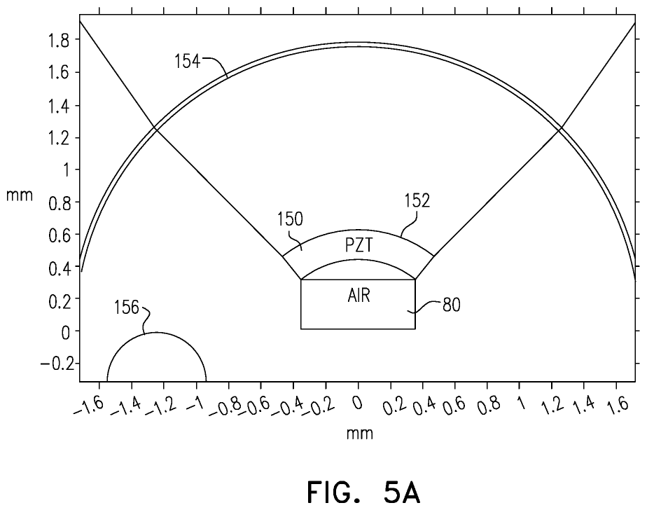

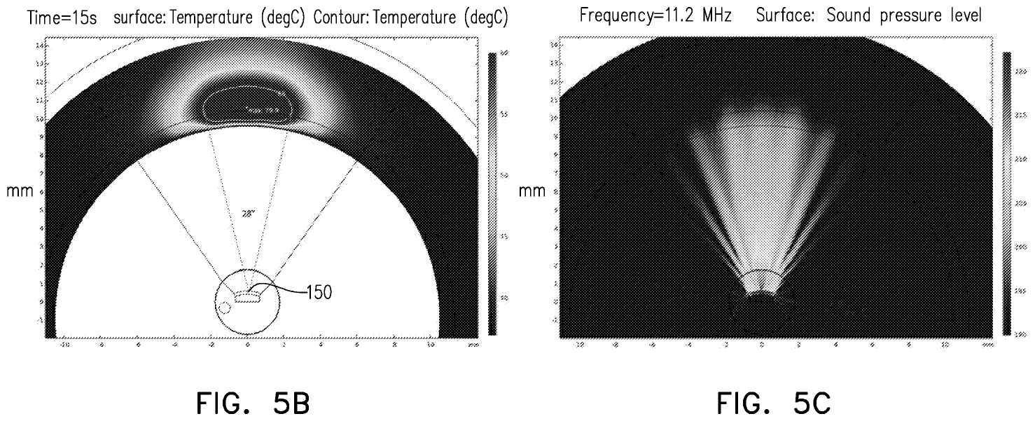

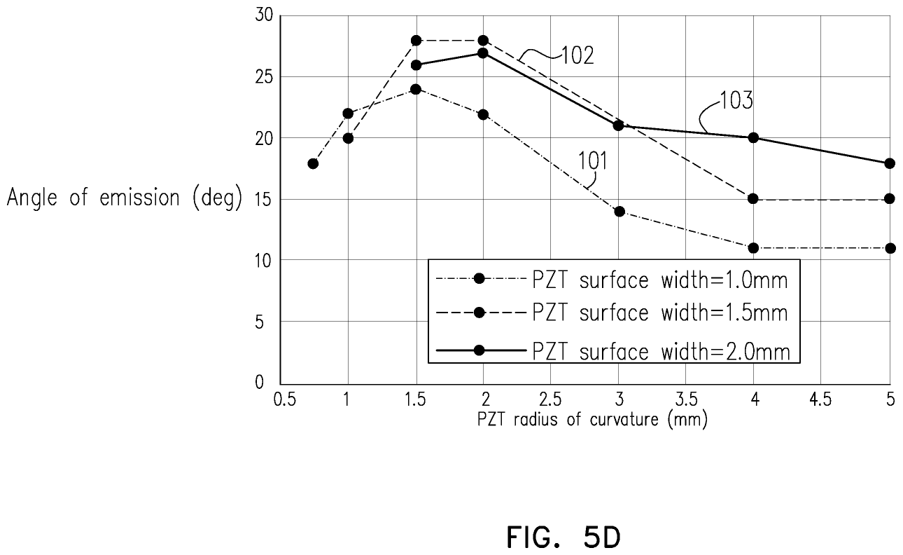

Typically, ultrasound transducer 50 is suspended over first support that comprises an air barrier (indicated by reference numeral 80 in FIG. 5A) allowing almost free vibrating of ultrasound transducer 50 with relatively low damping during application of the ablative energy to the tissue. This typically enables effective transmission of the ablative energy to the tissue. In contrast, pulse-echo ultrasound transducer 52 that detects the return signal (reflection) of the transmitted pulse-echo ultrasound energy as transducer 52, generally does not self-vibrate during detection of the reflected signal.

Since, as shown in FIG. 3A, transducers 50 and 52 are located adjacently to one another on ablation catheter 40 (and typically assembled within a single housing) there is a need to inhibit the mechanical vibrations of transducer 50 from reaching transducer 52 as vibration of transducer 52 is likely to render it less effective in detecting the reflected signal (in particular signals reflected from close proximity of the transducer (e.g., from a distance of 1 mm)).

Typically, in contrast to ultrasound transducer 50, which is suspended over an air barrier allowing almost free vibrating of ultrasound transducer 50 with relatively low damping during application of the ablative energy to the tissue, pulse-echo ultrasound transducer 52 is supported by a damping support that includes a backing layer 120 (FIG. 3B) and/or a damping element 140, e.g., damping ring (FIG. 3C), with relatively high damping. Thereby, sensing is enabled by ultrasound transducer 52 and ablative energy transmission is enabled by ultrasound transducer 50 while both transducers are located adjacent to each other. Typically, backing layer 120 is surrounded by damping element 140 which comprises soft material and/or high density (such as low durometer Pebax® or Pebax® and/or tungsten) intended to isolate mechanical vibrations of ultrasound transducer 50 from reaching ultrasound transducer 52. It is noted that damping element 140 is shown having a round shape in FIG. 3C by way of illustration and not limitation. Damping element 140 is typically shaped to correspond to the shape of ultrasound transducer 52 (and, for example, may have a rectangular or a square shape).

As described hereinabove, for some applications, the reflected ultrasound energy can be indicative of the ultrasound energy applied to the tissue and an effect of the ultrasound energy on the tissue and can serve as input for altering operational parameters of the ultrasound transducers. For example, the power, duty cycle, or any other parameter of the ablation are modulated in response to the detected reflected ultrasound energy. For some applications, the reflected ultrasound energy is used to assess the outcome of an ablation treatment post-treatment, and/or to assess lesion formation progression during an ablation treatment.

For some applications, the computer processor 26 is configured detect an indication of blood charring in the vicinity of the ultrasound transducers 50 and/or 52 by determining a parameter of the reflected pulse-echo ultrasound energy and inhibit application of ultrasound energy from the ultrasound transducer in response to the detected indication of blood charring. Typically, since blood charring blocks transmission of ultrasound, and the distance between the transducer and the tissue is known, indication of blood charring and a location thereof can be derived from parameters of the reflected ultrasound energy.

Reference is again made to FIGS. 1A-3A, showing apparatus 20, in accordance with some applications of the present invention. As described hereinabove, transluminal ablation catheter 40 has an elongated shaft comprising a handle 25 at the proximal portion of the elongated shaft and at least one ultrasound transducer 50 at the distal portion of the elongated shaft.

Typically, ultrasound transducer 50 is rotatable with respect to longitudinal axis LA of ablation catheter 40 in the direction indicated by arrow A1, in FIG. 1B. Rotation of ultrasound transducer 50 (and/or transducer 52) generally facilitates various functions and operational features of apparatus 20.

For example, ultrasound transducer 50 (and/or transducer 52) rotates to generate an image of the tissue. The images may be two-dimensional images and/or three-dimensional images. The images that are generated may be displayed on display 23 shown in FIG. 1A. For generating a three-dimensional image, ultrasound transducer 50 (and/or transducer 52) is both rotated (as indicated by arrow A1 in FIG. 1A) and translated back and forth longitudinally along axis LA (as indicated by arrow A2 in FIG. 1A). For some applications, transducer 50 (and/or transducer 52) provides continuous imaging of the anatomical structure in which transducer 50 (and/or transducer 52) is positioned (e.g., a chamber of the heart and an ostium of the lumen extending from the chamber). Typically, generating the three-dimensional image of the anatomical structure enables identifying an optimal location in the tissue and an optimal plane in which to perform the ablation. Typically, transducer 50 (and/or transducer 52) additionally, provides imaging of the vicinity of the anatomical structure, which is designated for ablation. For example, using imaging, a location the esophagus can be identified with respect to apparatus 20, in order to reduce potential damage to the esophagus that may be caused by ablation procedures performed on the heart.

Additionally, or alternatively, ultrasound transducer 50 rotates to aim transducer 50 at a tissue site that is designated for ablation/imaging. Further additionally, or alternatively, ultrasound transducer 50 may be rotated while continuously transmitting ablating ultrasound energy, thus creating a continuous circular lesion surrounding the ostium of the lumen of the blood vessel (e.g., the pulmonary vein ostium).

Typically, rotation of ultrasound transducer 50 (and/or transducer 52) occurs due to rotation of the elongated shaft of ablation catheter 40 to which ultrasound transducer 50 (and/or transducer 52) is coupled. Rotation of the elongated shaft of ablation catheter 40 is caused by rotation of an actuator (e.g., a knob or a motor) at the proximal portion of the elongated shaft (e.g., at handle 25, shown in FIG. 1A). The rotational motion is transmitted from the proximal portion along the elongated shaft to the distal portion of the shaft to which ultrasound transducer 50 (and/or transducer 52) is coupled, such as to rotate ultrasound transducer 50 (and/or transducer 52).

It is generally advantageous to determine the rotational and longitudinal position of ultrasound transducer 50 and/or transducer 52. For example, monitoring the rotational angle of ultrasound transducer 50 (and/or transducer 52) typically facilitates creating smooth two- and three-dimensional images. Since the rotational motion is generated in the proximal portion of the elongated shaft (e.g., at the handle), it is possible to measure the rotation angle of the handle to assess the rotation angle of ultrasound transducer 50 (and/or transducer 52). However, it may be the case that not all of the rotational motion originating in the proximal portion of the elongated shaft is transmitted along the elongated shaft to the distal portion of the shaft. Additionally, there may be a lag between a rotational position of the proximal portion of the elongated shaft and the distal portion of the of the elongated shaft. Therefore, when it is desirable to accurately determine a rotational position of ultrasound transducer 50 (and/or transducer 52, e.g., to determine the angle of rotation of ultrasound transducer 50 and/or transducer 52), it may not be sufficient to measure the rotation angle of the proximal portion of the elongated shaft (e.g., at the handle), at which the rotation starts.

In accordance with some applications of the present invention, apparatus 20 comprises mechanisms configured to determine the rotational position of the distal portion of the elongated shaft, and thus determine the rotational position of ultrasound transducer 50 (and/or transducer 52), independently of the rotational position of the proximal portion of the elongated shaft.

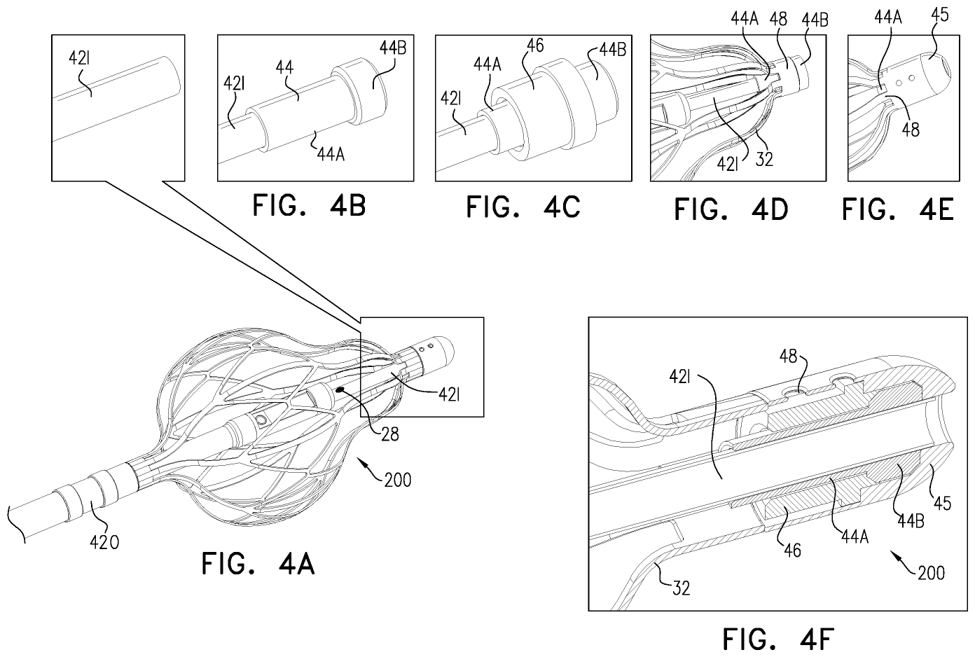

Accordingly, for some applications, apparatus 20 comprises a distal rotation detection sensor 28 (illustrated schematically in FIG. 4A).

For example, a gyroscope is coupled to ultrasound transducer 50 (and/or transducer 52). The gyroscope is typically configured to measure the rotational angle of the distal portion of the elongated shaft, and thus determine the rotational angle of ultrasound transducer 50 (and/or transducer 52).

Additionally, or alternatively, the rotational position of the distal portion of the elongated shaft may be determined using image-guide techniques, e.g., utilizing fiducial markers (not shown) that are coupled to expandable cage 30 and that reflect ultrasound energy that is emitted by ultrasound transducer 50 (and/or transducer 52). As described hereinbelow, expandable cage 30 typically remains stationary during rotation of the distal portion of the elongated shaft and transducer 50 (and/or transducer 52). Therefore, for some applications, the rotational position of ultrasound transducer 50 (and/or transducer 52) relative to the stationary cage is determined by identifying the positions of the fiducial markers within ultrasound images that are generated using ultrasound transducer 50 (and/or transducer 52), and thereby deriving the rotational position of ultrasound transducer 50 (and/or transducer 52) relative to expandable cage 30.

Further additionally, or alternatively, the rotational position of the distal portion of the elongated shaft may be determined using any other type of one or more sensors disposed at the distal portion of the shaft, on expandable cage 30, and/or on ultrasound transducer 50. For example, a first magnetic coil may be coupled to the distal portion of the shaft and/or to ultrasound transducer 50 (and/or transducer 52), such that the rotational position of the first magnetic coil changes as the rotational position of the ultrasound transducer 50 (and/or transducer 52) changes. A second magnetic coil may be coupled to the cage, such that the rotational position of the second magnetic coils remains constant as ultrasound transducer 50 (and/or transducer 52) rotates. Additionally, or alternatively, the second magnetic coil may be coupled to the distal portion of the shaft in a manner such that the rotational position of the second magnetic coil remains constant as ultrasound transducer 50 (and/or transducer 52) rotates. The rotational position of ultrasound transducer 50 (and/or transducer 52) is derived by measuring a change in magnetic flux between the first and second coils.

As described hereinabove, both expandable cage 30 and ultrasound transducer 50 are disposed at the distal end of the elongated shaft of transluminal ablation catheter 40. Although expandable cage 30 is coupled to the shaft, expandable cage 30 remains both expanded and stationary during rotation the distal portion of the shaft and ultrasound transducer 50. (Rotational space for expandable cage 30 in the lumen is limited, thereby generally preventing the ability of expandable cage 30 to rotate. Therefore, attempting rotation of expandable cage 30, is likely to inhibit rotation of ultrasound transducer 50). In order to maintain expandable cage 30 stationary during of rotation of the elongated shaft, for some applications, apparatus 20 comprises a rotational-force reduction mechanism for reducing rotational force applied to expandable cage 30 by the elongated shaft upon rotation of the elongated shaft, such as to hold the expandable cage 30 stationary during rotation of ultrasound transducer 50.

Reference is now made to FIGS. 4A-F, which are schematic illustrations of components of a rotational-force reduction mechanism 200 configured to reduce rotational force applied to expandable cage 30 by elongated shaft 42 of transluminal ablation catheter 40 upon rotation of the elongated shaft. Components of rotational-force reduction mechanism 200 are typically disposed at the distal portion of elongated shaft 42, allowing rotation of ultrasound transducer 50, while holding expandable cage 30 stationary, similar to a swivel mechanism. For some such applications, elongated shaft 42 comprises an inner elongated shaft 421 and an outer elongated shaft 420. Typically, the proximal end of expandable cage 30 is coupled to outer elongate shaft 420. Further typically, ultrasound transducer 50 is disposed on inner elongated shaft 421, and rotation is transmitted to the ultrasound transducer via rotation of inner elongated shaft 421 within outer elongated shaft 420 (i.e., inner elongated shaft 421 rotates within outer elongated shaft 420, while outer elongated shaft remains rotationally stationary).

For some applications, rotational-force reduction mechanism 200 comprises a swivel bearing 44, a bearing stopper 46 and a distal tip cover 45 (FIGS. 4B, 4C, 4D, 4E, and 4F). Typically, the swivel bearing includes a narrow proximal portion 44A, and a distal wider portion 44B, which has a greater diameter than the proximal portion. Swivel bearing 44 is typically coupled to the distal portion of inner elongated shaft 421 (FIG. 4B), such that it rotates with elongated shaft 42. Bearing stopper 46 is placed to surround the proximal narrow portion of swivel bearing 44, and such that distal wide portion of the swivel bearing is disposed distally to the bearing stopper. The diameter of the distal wide portion of the swivel bearing is typically greater than a lumen defined by the bearing stopper, such that the distal wide portion of the swivel bearing prevents the swivel bearing from being pulled proximally with respect to the bearing stopper. Distal tip cover 45 typically covers distal wide portion 44B of the swivel bearing. The bearing stopper and distal tip cover are configured to allow continuous and smooth rotation of the swivel bearing (and thereby allow continuous and smooth rotation of the elongated shaft), while the bearing stopper and distal tip cover are held rotationally stationary.

Typically, expandable cage 30 is inhibited from rotating (a) by the proximal end of the cage being coupled to outer elongated shaft 420 and (b) by the cage itself contacting tissue of the subject as described hereinabove. A distal portion of the cage (e.g., cage distal ring 48) is typically coupled to bearing stopper 46, and thereby applies torsional forces to the bearing stopper that inhibit the bearing stopper from rotating. The bearing stopper typically allows inner elongated shaft 421 to continue to rotate due to swivel bearing 44 being able to rotate freely within the bearing stopper and separates the rotational motion of the inner elongated shaft from the cage, such as to prevent any torsional forces from being applied to cage 30.

Swivel bearing 44 typically allows rotation of elongated shaft 42 while still allowing expanding and collapsing of expandable cage 30 at the distal end of elongated shaft 42. As described hereinabove, typically, the proximal end of expandable cage 30 is coupled to outer elongated shaft 420. Further typically, in order to radially expand the cage, the distal end of the cage is pulled proximally toward the proximal end of the cage, by pulling the distal end of inner elongated shaft 421 proximally toward the outer elongated shaft 420. Wide distal portion 44B typically transmits the proximal motion of the distal end of the inner elongated shaft to the bearing stopper, which in turn transmits the proximal motion to the distal end of the cage, thereby pulling the distal end of the cage toward the proximal end of the cage to axially shorten the cage and radially expand the cage. Typically, in order to radially contract the cage, the distal end of the cage is pushed distally away from the proximal end of the cage, by pushing the distal end of inner elongated shaft 421 distally away from the outer elongated shaft 420. Wide distal portion 44B typically transmits the distal motion of the distal end of the inner elongated shaft to distal tip cover 45, which in turn transmits the distal motion to bearing stopper and the distal end of the cage, thereby pushing the distal end of the cage away from the proximal end of the cage to axially elongate the cage and radially contract the cage.

Thus, rotational-force reduction mechanism 200 is configured on the one hand to couple axial motion of inner elongated shaft 421 to axial motion of the distal end of cage 30, but on the other hand to separate between rotational motion of inner elongated shaft 421 and rotational motion of the distal end of cage 30.

FIG. 4F is a cross section of the components of rotational-force reduction mechanism 200 assembled with apparatus 20 to inhibit rotation of expandable cage 30 during rotation of ultrasound transducer 50, as described hereinabove with reference to FIGS. 4A-E.

Reference is now made to FIGS. 5A, 5B and 5C, which are schematic illustrations of a curved piezoelectric ultrasound transducer 150, and ultrasound energy transmission profiles thereof, in accordance with some applications of the present invention. Reference is also made to FIG. 5D, which is a graph showing the effect of various radii of curvature and lengths of piezoelectric ultrasound transducer 150 on an angle of emission of piezoelectric ultrasound transducer 150, in accordance with some applications of the present invention.

As shown in FIG. 5A, for some applications, ultrasound transducer 50 comprises a curved piezoelectric ultrasound transducer 150. FIG. 5A represents a cross section of curved piezoelectric ultrasound transducer 150, suspended over air barrier 80, and placed within a transducer housing encapsulation layer 154, in the vicinity of a cooling channel 156.

Piezoelectric ultrasound transducer 150 is shaped to define a convex surface 152 facing outwardly from a longitudinal axis of the transducer such that the convex surface has a radius of curvature of 0.75-5 mm. Typically, piezoelectric ultrasound transducer 150 is configured to ablate tissue (e.g., of an ostium of the pulmonary vein) by applying ultrasound energy to the tissue (e.g., of the ostium) from the convex surface. Typically, due to the curvature of transducer 150, the area that is affected by the ultrasound energy is increased (compared to use of a flat transducer), allowing for a faster and/or more efficient ablation procedure.

For some applications, ultrasound transducer 150 has a width of 0.5-3 mm (e.g., 1-2 mm), and a thickness of 0.1-0.3 mm. For some applications, piezoelectric ultrasound transducer 150 has a radius of curvature of 0.75-5 mm, e.g., 1-3 mm, e.g., 1.5-2 mm.

FIGS. 5B and 5C represent thermal (FIG. 5B) and pressure (5C) profiles within pulmonary veins, when performing ablation with curved piezoelectric ultrasound transducer 150, in accordance with some application of the present invention. Typically, providing curved piezoelectric ultrasound transducer 150 allows to widen a thermal profile of the tissue such that a larger portion of the tissue is heated more effectively, thereby facilitating effective and more rapid ablation of the tissue. In some cases, the heated tissue area is at least two times greater when using curved piezoelectric ultrasound transducer 150, in comparison to using a flat piezoelectric ultrasound transducer of the same width, length, and thickness and while applying the same power parameters. As shown in FIG. 5B, providing piezoelectric ultrasound transducer 150 having a radius of curvature of 2 mm, and a width of 1.5 mm resulted in an angle of emission of 28 degrees, and a generally uniform thermal profile of the tissue, generally without the presentation of side lobes (indicative of a generally homogeneous lesion).

The graph in FIG. 5D shows the effect of the radius of curvature and the surface widths of piezoelectric ultrasound transducer 150 on an angle of emission of piezoelectric ultrasound transducer 150, in accordance with some applications of the present invention. In FIG. 5D, line 101 shows the effect of the radius of curvature on the angle of emission using a piezoelectric ultrasound transducer having a surface width of 1 mm. Line 102 shows the effect of the radius of curvature on the angle of emission using a piezoelectric ultrasound transducer having a surface width of 1.5 mm, and line 103 shows the effect of the radius of curvature on the angle of emission using a piezoelectric ultrasound transducer having a surface width of 2 mm. As shown, providing ultrasound transducer 150 having a radius of curvature of 1.5-2 mm, and a surface width of 1.5 mm resulted in an angle of emission of 28 degrees (indicated by line 102).

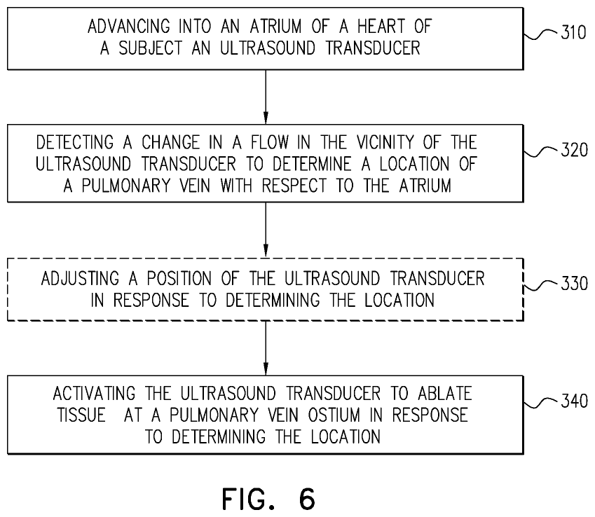

Reference is again made to FIGS. 1A-5A, and to FIG. 6. For some applications, a sensor, in operable communication with transluminal ablation catheter 40 (e.g., coupled to catheter 40), is used to detect a change in blood flow in the between the lumen of the subject that extends from the chamber of a heart and the chamber of the heart (e.g., change in blood flow between the pulmonary vein and the atrium), indicating the connection point between the lumen and the heart chamber, thereby indicating the location of the ostium of the lumen. Typically, a change in blood flow can be detected when blood passes through the ostium of the lumen and into the chamber (e.g., the ostium of the pulmonary vein and the atrium). Based on the detected change in the blood flow, computer processor 26 is configured to determine an optimal tissue target site for ablation (e.g., the ostium of the lumen), and drive ultrasound transducer 50 and/or 150 to transmit the ultrasound energy to the optimal tissue target.

For some applications, the sensor comprises a Doppler sonography device to sense the blood flow and detect changes in the pattern in the blood flow. Additionally, or alternatively, transluminal ablation catheter 40 further comprises an actuator configured to adjust a position of ultrasound transducer 50 and/or 150 in response to the detected change in flow, so that the transmitted ultrasound energy is applied to the optimal tissue target site.

Reference is made to FIG. 6, which is a flow chart showing steps of a method performed with respect to detecting changes in blood flow, in accordance with some applications of the present invention. For some applications, ultrasound transducer 50 and/or 150 is advanced into an atrium of a heart of a subject (step 310), a change in blood flow in the vicinity of the ultrasound transducer is detected to determine a location of a pulmonary vein with respect to the atrium (step 320), and ultrasound transducer 50 and/or 150 are activated to ablate tissue at a pulmonary vein ostium in response to determining the location (step 340). Optionally, prior to activation the ultrasound transducer (step 340), a position of ultrasound transducer 50 and/or 150 is adjusted in response to determining the location (step 330).

For some applications, the location data derived from the change in flow detected by the Doppler sonography is used in combination with three-dimensional image data of the atrium and the pulmonary vein ostium in order to verify the location of the pulmonary vein ostium with respect to the atrium.

In accordance with some applications of the present invention, any type of sound indication may be used to detect the change of the blood flow pattern between the atrium and the pulmonary vein to indicate the location of the pulmonary vein ostium, which is typically the desired ablation site. For example, any one of ultrasound transducers 50, 52, and/or 150 described herein may be used to detect the change of the blood flow pattern between the atrium and the pulmonary vein.

Reference is again made to FIGS. 1A-6. As described hereinabove, apparatus 20 is configured to perform acoustic sensing in addition to ablating and imaging tissue of the myocardial tissue.

For example, prior to transmission of ultrasound energy for ablation purposes, apparatus 20 is configured to assess whether ultrasound transducer 50 is optimally positioned with respect to the target tissue designated for ablation by means of acoustic sensing, and in response apply ablating energy to the tissue, or alternatively, adjust a position of ultrasound transducer 50 and/or adjust the energy level applied to the tissue.

For some applications, apparatus 20 (in particular, ultrasound transducers 50/52/150 of apparatus 20), is configured to transmit low intensity, non-ablating ultrasound energy in order to verify proper positioning the ultrasound transducers. For some applications, apparatus 20 assesses the proper positioning of ultrasound transducers 50/52/150 with respect to the target tissue by assessing a parameter of the ultrasound echo received by the transducers. For example, apparatus 20 assesses the proper positioning of ultrasound transducers 50/52/150 with respect to the target tissue by measuring the amplitude of the ultrasound echo received by the transducers (i.e., the energy level of the echo). The amplitude of the echo is small if ultrasound transducers 50/52/150 are mispositioned with respect to the tissue (because the energy delivered is dispersed and not well directed due to angulation of the acoustic field with the tissue that creates a large contact area and therefore low energy density) and increases when ultrasound transducers 50/52/150 are properly positioned with respect to the target tissue. Typically, in response to assessing that ultrasound transducers 50/52/150 is in the desired position, the ultrasound transducers are activated to ablate the tissue. Alternatively, the energy level applied to the tissue is adjusted to compensate for suboptimal positioning of ultrasound transducers 50/52/150. Further alternatively, a position of ultrasound transducers 50/52/150 is adjusted to better target the tissue.

Additionally, or alternatively, when applying the ablating energy to form lesions in the tissue, the ablation can be monitored by measuring the amplitude of the return ultrasound echo received by the transducers from the target tissue. Since the ultrasound energy does not transmit through the lesion, an increase in the return ultrasound is indicative of a well-formed lesion in the tissue.

Additionally, or alternatively, measuring the amplitude (or another parameter) of the ultrasound echo returning from different tissue depths is used to compare and assess changes (e.g., formation of lesions) along the depth of the tissue. For some applications, a graphical representation of the return ultrasound echo is displayed. For example, a graphical representation may be presented in which each depth interval is assigned a color pixel indicating the extent to which that depth interval has been ablated (e.g., at a resolution of 0.1-0.3 mm).

Reference is still made to FIGS. 1A-6. It is noted that in accordance with some applications of the present invention, apparatus 20 can be used to treat types of cardiac arrhythmia other than atrial fibrillation. For example, apparatus 20 is used to treat conditions such as ventricular tachycardia. For such applications, apparatus 20 is advanced into a ventricle of a subject and lesions are created by ablation of tissue in the ventricle by application of ultrasound energy in accordance with applications of the present invention.

It is further noted that application of ultrasound energy to myocardial sites is not limited to blood vessel orifices but may be applied to any region in the heart which is involved in triggering or maintaining cardiac arrhythmias.

It is further noted that, although much of the description herein relates to cardiac tissue, particularly, the left atrium and pulmonary veins extending from the atrium, the scope of the present invention includes the use of the apparatus and methods described herein with respect to other lumens in the body (“lumen” generally referring to an inner open space, i.e., a cavity, within an organ in a subject's body, which may be, but is not necessarily, a tubular organ). For example, the apparatus and methods described herein may be used, mutatis mutandis, with respect to an artery, vein, intestine, heart, bladder, sinus, stomach, lungs, lung vasculature, respiratory tract of the subject or urogenital tract of the subject.

It is further noted that apparatus and methods described herein may additionally be used, mutatis mutandis, to treat other tissue of a subject for a treatment that includes renal denervation, targeted lung denervation, pulmonary hypertension denervation, splanchnic nerve denervation, carotid body denervation, cancerous lung nodule ablation, hypertrophic cardiomyopathy ablation, and/or hepatic artery denervation.

For some applications, the ultrasound energy application techniques described herein are practiced in combination with other types of ablation, such as Pulsed Field Ablation (PFA) and/or radiofrequency (RF) ablation. For some applications, other suitable energy sources (e.g., RF, laser, cryogenic, and/or electromagnetic energy such as ultraviolet and/or infrared) are used as an alternative or in addition to ultrasound ablation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for use with tissue of a subject, the apparatus comprising:
a transluminal ablation catheter comprising:
at least one ultrasound transducer configured to be inserted into a chamber of the subject's heart, and: (a) to ablate tissue of the subject by applying ultrasound energy to the tissue, and (b) to image tissue of the subject by applying non-ablating ultrasound energy to the tissue; and
an expandable element configured to be disposed around the at least one ultrasound transducer,
the at least one ultrasound transducer being configured to rotate and axially translate back and forth within the expandable element, such as to generate a three-dimensional image of the tissue.

2. The apparatus according to claim 1, wherein the at least one ultrasound transducer is configured to be inserted into a left atrium in a vicinity of a pulmonary vein ostium and is configured to ablate tissue of the pulmonary vein ostium, to thereby electrically isolate the pulmonary vein.

3. The apparatus according to claim 1, wherein the tissue includes tissue of an ostium of a lumen that extends from the chamber of the subject's heart, and wherein the ultrasound transducer is configured to generate a three-dimensional image of the tissue of the ostium of the lumen by applying non-ablative ultrasound energy to the tissue of the ostium of the lumen.

4. The apparatus according to claim 1, wherein the tissue includes tissue of an ostium of a lumen that extends from the chamber of the subject's heart, wherein the expandable element comprises an expandable cage that comprises a plurality of struts, at least a portion of the struts being curved outwardly at at least two locations along the strut, such that the cage is configured to temporarily anchor a distal portion of the transluminal ablation catheter in the lumen by the portion of the struts contacting a wall of the lumen.

5. The apparatus according to claim 1, wherein the tissue includes tissue of an ostium of a lumen that extends from the chamber of the subject's heart, and wherein the expandable element comprises an expandable cage has having a central portion and a distal portion and being shaped to define a nipple-like structure by the central portion having a diameter that is greater than a diameter of the distal portion such that the distal portion is shaped and sized to be inserted into an ostium of the lumen to temporarily anchor the distal in the lumen by the contacting a wall of the lumen

6. The apparatus according to claim 1, wherein the at least one ultrasound transducer is configured to generate ultrasound energy at a frequency of 8-20 MHz.

7. The apparatus according to claim 1, wherein the at least one ultrasound transducer is shaped to define a convex surface facing outwardly from a longitudinal axis of the transducer, and having a width of 0.5-3 mm and a radius of curvature of 0.75-5 mm.

8. The apparatus according to claim 1, wherein the tissue includes tissue of an ostium of a lumen that extends from the chamber of the subject's heart, and wherein the expandable element comprises an expandable cage that comprises a plurality of struts, and at least a portion of the plurality of struts comprise electrically conductive struts that are configured to contact tissue of an ostium of the lumen and to ablate tissue of the ostium of the lumen that is in contact with the electrically conductive struts by driving current into the tissue of the ostium of the lumen.

9. The apparatus according to claim 8, wherein at least a portion of the electrically conductive struts comprise an insulated portion and an electrically conductive portion, and wherein the electrically conductive portion is configured to contact tissue of the ostium of the lumen and to ablate tissue of the ostium of the lumen.

10. The apparatus according to claim 1, wherein the expandable element comprises an expandable cage that comprises a plurality of struts, and at least a portion of plurality of the struts are shaped to define an aperture formed in the strut through which ultrasound energy is transmitted from the ultrasound transducer to the tissue.

11. The apparatus according to claim 10, wherein within the portion of plurality of the struts each of the struts has a width of 0.5-1 mm, and the aperture in the strut has a width of 0.25-0.5 mm.

12. The apparatus according to claim 10, wherein a thickness of the strut is 0.1-0.25 mm.

13. The apparatus according to claim 1, wherein the transluminal ablation catheter comprises an elongated shaft comprising a proximal portion comprising a handle, and a distal portion to which the at least one ultrasound transducer is coupled.

14. The apparatus according to claim 13, wherein the elongated shaft is configured to be rotatable, such as to rotate the ultrasound transducer, and the transluminal ablation catheter comprises one or more sensors coupled to the distal portion of the elongated shaft and configured to detect a rotational position of the distal portion of the elongated shaft.

15. The apparatus according to claim 13, wherein:
the expandable element comprises an expandable cage;
the elongated shaft is configured to be rotatable, such as to rotate the ultrasound transducer; and
the transluminal ablation catheter further comprises a rotational-force reduction mechanism configured to reduce rotational force applied to the expandable cage by the elongated shaft upon rotation of the elongated shaft, such as to hold the expandable cage stationary during rotation of the ultrasound transducer.

16. The apparatus according to claim 1,
wherein the at least one ultrasound transducer is configured to apply the non-ablating ultrasound energy to the tissue such that at least a portion of the non-ablating ultrasound energy is reflected and received by the ultrasound transducer; and
wherein the apparatus further comprises a computer processor configured to assess a parameter of the reflected energy to determine a parameter of the ultrasound energy to be applied by the ultrasound transducer to ablate the tissue; and
wherein the at least one ultrasound transducer is configured to apply the ultrasound energy to the tissue based on the determined parameter.

17. The apparatus according to claim 1, further comprising an inflatable element configured to be disposed around the ultrasound transducer.

18. (canceled)

19. The apparatus according to claim 1, wherein the at least one ultrasound transducer comprises:
a first ultrasound transducer configured to ablate the tissue of the subject by transmitting ablative ultrasound energy toward the tissue; and
a second ultrasound transducer configured to image the tissue of the subject by transmitting one or more pulses of pulse-echo ultrasound energy toward the tissue and receiving a reflection of the transmitted pulse-echo ultrasound energy, and the second ultrasound transducer is configured to rotate and axially translate back and forth within the expandable element, such as to generate a three-dimensional image of the tissue.

20. The apparatus according to claim 19, wherein the transluminal ablation catheter comprises:
a first support configured to support the first ultrasound transducer and enable transmitting of the ablative ultrasound energy toward the tissue; and
a second damping support that is configured to support the second ultrasound transducer and provide a higher level of damping than damping provided by the first support, such as to enable the second ultrasound transducer to receive the reflection of the transmitted pulse-echo ultrasound energy, while the first ultrasound transducer is transmitting the ablative ultrasound energy toward the tissue.

21. The apparatus according to claim 20, wherein:
the first support comprises an air barrier configured to allow vibration of the first ultrasound transducer during transmitting of the ablative ultrasound energy toward the tissue; and
the second ultrasound damping support comprises a mechanical support comprising at least one of a backing layer and a damping element.

22-56. (canceled)

57. A method for use with tissue of a subject, the method comprising:
inserting a transluminal ablation catheter into a chamber of the subject's heart, the transluminal ablation catheter including one or more ultrasound transducers, and an expandable element disposed around the one or more ultrasound transducers;
ablating tissue of the subject by applying ultrasound energy to the tissue, using the one or more ultrasound transducers;
imaging tissue of the subject by applying non-ablating ultrasound energy to the tissue, using the one or more ultrasound transducers; and
while one of the one or more ultrasound transducers is imaging the tissue of the subject, rotating and axially translating the one of the one or more ultrasound transducers, such as to generate a three-dimensional image of the tissue.
